SEQUENCE LISTING (35 sequences)

SEQ ID NO: 1 - DNA, 3949 bp, Homo sapiens
gcggtggcgcgggagcggccccggggaccccgcgctgctgacggcggcgaccgcggccggaggcgggcgcgggtctcggaggcggtcgcctcagcaccgccccacgggcggccccagcccctcccgcagccctcctccctcccgctcccttcccgccgcctcctccccgccctcctccctcctcgctcgcgggccgggcccggcatggtgcggcgtcgccgccgatggcgctgaggcggagcatggggcggccggggctcccgccgctgccgctgccgccgccaccgcggctcgggctgctgctggcggctctggcttctctgctgctcccggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgcgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgttagcccacaggcagagggcatcggggccatttggccggctctggtggccactgagctggctgactaagccccgtctgaccccagcccagacagcaaggtgtggaggctcctgtggtagtcctcccaagctgtgctgggaagcccggactgaccaaatcacccaatcccagttcttcctgcaaccactctgtggccagcctggcatcagtttaggccttggcttgatggaagtgggccagtcctggttgtctgaacccaggcagctggcaggagtggggtggttatgtttccatggttaccatgggtgtggatggcagtgtggggagggcaggtccagctctgtgggccctaccctcctgctgagctgcccctgctgcttaagtgcatgcattgagctgcctccagcctggtggcccagctattaccacacttggggtttaaatatccaggtgtgcccctccaagtcacaaagagatgtccttgtaatattcccttttaggtgagggttggtaaggggttggtatctcaggtctgaatcttcaccatctttctgattccgcaccctgcctacgccaggagaagttgaggggagcatgcttccctgcagctgaccgggtcacacaaaggcatgctggagtacccagcctatcaggtgcccctcttccaaaggcagcgtgccgagccagcaagaggaaggggtgctgtgaggcttgcccaggagcaagtgaggccggagaggagttcaggaacccttctccatacccacaatctgagcacgctaccaaatctcaaaatatcctaagactaacaaaggcagctgtgtctgagcccaacccttctaaacggtgacctttagtgccaacttcccctctaactggacagcctcttctgtcccaagtctccagagagaaatcaggcctgatgagggggaattcctggaacctggaccccagccttggtgggggagcctctggaatgcatggggcgggtcctagctgttagggacatttccaagctgttagttgctgtttaaaatagaaataaaattgaagact

SEQ ID NO: 2 - protein, 890 aa, Homo sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 3 - protein, 678 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 4 - DNA, 2461 bp, Homo sapiens
ccgcagccgccgccgccgccgccgccgcgatgtgaccttcagggccgccaggacgggatgaccggagcctccgccccgcggcgcccgctcgcctcggcctcccgggcgctctgaccgcgcgtccccggcccgccatggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaat

SEQ ID NO: 5 - DNA, 21 bp, Artificial
  PCR primer
tttccctggcaaggactatga

SEQ ID NO: 6 - DNA, 17 bp, Artificial
  PCR primer
aatggcgtgagtcgggc

SEQ ID NO: 7 - DNA, 26 bp, Artificial
  PCR primer
tgatctcttttggaattaaggagcat

SEQ ID NO: 8 - DNA, 23 bp, Artificial
  PCR primer
atgggcatctcctccataatttg

SEQ ID NO: 9 - DNA, 19 bp, Artificial
  PCR primer
aaattgctggcagggttgc

SEQ ID NO: 10 - DNA, 21 bp, Artificial
  PCR primer
tttctgtactgcgggtggaac

SEQ ID NO: 11 - DNA, 19 bp, Artificial
  PCR primer
gcaaaccttcaaggcagcc

SEQ ID NO: 12 - DNA, 19 bp, Artificial
  PCR primer
tgctgtttgcctcggacat

SEQ ID NO: 13 - DNA, 16 bp, Artificial
  PCR primer
acgctgctcgtcgccg

SEQ ID NO: 14 - DNA, 20 bp, Artificial
  PCR primer
gccagcctcctggacatcct

SEQ ID NO: 15 - DNA, 23 bp, Artificial
  PCR primer
acccaacaccaagacacagttct

SEQ ID NO: 16 - DNA, 33 bp, Artificial
  PCR primer
tcttactgctatacctttactctttatggtgta

SEQ ID NO: 17 - DNA, 19 bp, Artificial
  PCR primer
cagccgcttcacctacagc

SEQ ID NO: 18 - DNA, 25 bp, Artificial
  PCR primer
ttttgtattcaatcactgtcttgcc

SEQ ID NO: 19 - DNA, 20 bp, Artificial
  PCR primer
gccagcctcctggacatcct

SEQ ID NO: 20 - DNA, 23 bp, Artificial
  PCR primer
agtcctttcaggctagctgcatc

SEQ ID NO: 21 - DNA, 17 bp, Artificial
  PCR primer
tcgaggacagcgaggcc

SEQ ID NO: 22 - DNA, 22 bp, Artificial
  PCR primer
tcgagggtgtagcgtgtagaga

SEQ ID NO: 23 - DNA, 19 bp, Artificial
  PCR primer
atggggaaggtgaaggtcg

SEQ ID NO: 24 - DNA, 20 bp, Artificial
  PCR primer
taaaagcagccctggtgacc

SEQ ID NO: 25 - RNA, 21 nt, Artificial
  siRNA
aaccaagugccacaaaggaac

SEQ ID NO: 26 - RNA, 21 nt, Artificial
  siRNA
aaacaucgagagagcugagga

SEQ ID NO: 27 - RNA, 21 nt, Artificial
  siRNA
aagugcgcugcuggugccaac

SEQ ID NO: 28 - DNA, 3309 bp, Homo sapiens
ctgcagggggggggggggggggggggggggggggggggcgcagcacggctcagaccgaggcgcacaggctcgcagctccgggcgcctagcgcccggtccccgccgcgacgcgccaccgtccctgccggcgcctccgcgccttcgaaatgagggtcctgggtgggcgctgcggggcgccgctggcgtgtctcctcctagtgcttcccgtctcagaggcaaaccttctgtcaaagcaacaggcttcacaagtcctggttaggaagcgtcgtgcaaattctttacttgaagaaaccaaacagggtaatcttgaaagagaatgcatcgaagaactgtgcaataaagaagaagccagggaggtctttgaaaatgacccggaaacggattatttttatccaaaatacttagtttgtcttcgctcttttcaaactgggttattcactgctgcacgtcagtcaactaatgcttatcctgacctaagaagctgtgtcaatgccattccagaccagtgtagtcctctgccatgcaatgaagatggatatatgagctgcaaagatggaaaagcttcttttacttgcacttgtaaaccaggttggcaaggagaaaagtgtgaatttgacataaatgaatgcaaagatccctcaaatataaatggaggttgcagtcaaatttgtgataatacacctggaagttaccactgttcctgtaaaaatggttttgttatgctttcaaataagaaagattgtaaagatgtggatgaatgctctttgaagccaagcatttgtggcacagctgtgtgcaagaacatcccaggagattttgaatgtgaatgccccgaaggctacagatataatctcaaatcaaagtcttgtgaagatatagatgaatgctctgagaacatgtgtgctcagctttgtgtcaattaccctggaggttacacttgctattgtgatgggaagaaaggattcaaacttgcccaagatcagaagagttgtgaggttgtttcagtgtgccttcccttgaaccttgacacaaagtatgaattactttacttggcggagcagtttgcaggggttgttttatatttaaaatttcgtttgccagaaatcagcagattttcagcagaatttgatttccggacatatgattcagaaggcgtgatactgtacgcagaatctatcgatcactcagcgtggctcctgattgcacttcgtggtggaaagattgaagttcagcttaagaatgaacatacatccaaaatcacaactggaggtgatgttattaataatggtctatggaatatggtgtctgtggaagaattagaacatagtattagcattaaaatagctaaagaagctgtgatggatataaataaacctggacccctttttaagccggaaaatggattgctggaaaccaaagtatactttgcaggattccctcggaaagtggaaagtgaactcattaaaccgattaaccctcgtctagatggatgtatacgaagctggaatttgatgaagcaaggagcttctggaataaaggaaattattcaagaaaaacaaaataagcattgcctggttactgtggagaagggctcctactatcctggttctggaattgctcaatttcacatagattataataatgtatccagtgctgagggttggcatgtaaatgtgaccttgaatattcgtccatccacgggcactggtgttatgcttgccttggtttctggtaacaacacagtgccctttgctgtgtccttggtggactccacctctgaaaaatcacaggatattctgttatctgttgaaaatactgtaatatatcggatacaggccctaagtctatgttccgatcaacaatctcatctggaatttagagtcaacagaaacaatctggagttgtcgacaccacttaaaatagaaaccatctcccatgaagaccttcaaagacaacttgccgtcttggacaaagcaatgaaagcaaaagtggccacatacctgggtggccttccagatgttccattcagtgccacaccagtgaatgccttttataatggctgcatggaagtgaatattaatggtgtacagttggatctggatgaagccatttctaaacataatgatattagagctcactcatgtccatcagtttggaaaaagacaaagaattcttaaggcatcttttctctgcttataataccttttccttgtgtgtaattatacttatgtttcaataacagctgaagggttttatttacaatgtgcagtctttgattattttgtggtcctttcctgggatttttaaaaggtcctttgtcaaggaaaaaaattctgttgtgatataaatcacagtaaagaaattcttacttctcttgctatctaagaatagtgaaaaataacaattttaaatttgaatttttttcctacaaatgacagtttcaatttttgtttgtaaaactaaattttaattttatcatcatgaactagtgtctaaatacctatgtttttttcagaaagcaaggaagtaaactcaaacaaaagtgcgtgtaattaaatactattaatcataggcagatactattttgtttatgtttttgtttttttcctgatgaaggcagaagagatggtggtctattaaatatgaattgaatggagggtcctaatgccttatttcaaaacaattcctcagggggaccagctttggcttcatctttctcttgtgtggcttcacatttaaaccagtatctttattgaattagaaaacaagtgggacatattttcctgagagcagcacaggaatcttcttcttggcagctgcagtctgtcaggatgagatatcagattaggttggataggtggggaaatctgaagtgggtacattttttaaattttgctgtgtgggtcacacaaggtctacattacaaaagacagaattcagggatggaaaggagaatgaacaaatgtgggagttcatagttttccttgaatccaacttttaattaccagagtaagttgccaaaatgtgattgttgaagtacaaaaggaactatgaaaaccagaacaaattttaacaaaaggacaaccacagagggatatagtgaatatcgtatcattgtaatcaaagaagtaaggaggtaagattgccacgtgcctgctggtactgtgatgcatttcaagtggcagttttatcacgtttgaatctaccattcatagccagatgtgtatcagatgtttcactgacagtttttaacaataaattcttttcactgtattttatatcacttataataaatcggtgtataatttt

SEQ ID NO: 29 - protein, 676 aa, Homo sapiens
MRVLGGRCGAPLACLLLVLPVSEANLLSKQQASQVLVRKRRANSLLEETKQGNLERECIEELCNKEEAREVFENDPETDYFYPKYLVCLRSFQTGLFTAARQSTNAYPDLRSCVNAIPDQCSPLPCNEDGYMSCKDGKASFTCTCKPGWQGEKCEFDINECKDPSNINGGCSQICDNTPGSYHCSCKNGFVMLSNKKDCKDVDECSLKPSICGTAVCKNIPGDFECECPEGYRYNLKSKSCEDIDECSENMCAQLCVNYPGGYTCYCDGKKGFKLAQDQKSCEVVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 30 - DNA, 4987 bp, Homo sapiens
gagtggagttctggaggaatgtttaccagacacagagcccagagggacagcgcccagagcccagatagagagacacggcctcactggctcagcaccagggtccccttccccctcctcagctccctccctggcccctttaagaaagagctgatcctctcctctcttgagttaacccctgattgtccaggtggcccctggctctggcctggtgggcggaggcaaagggggagccaggggcggagaaagggttgcccaagtctgggagtgagggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccagcaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctacttttttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctgcattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttcccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 31 - protein, 885 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 32 - DNA, 5014 bp, Homo sapiens
gagtggagttctggaggaatgtttaccagacacagagcccagagggacagcgcccagagcccagatagagagacacggcctcactggctcagcaccagggtccccttccccctcctcagctccctccctggcccctttaagaaagagctgatcctctcctctcttgagttaacccctgattgtccaggtggcccctggctctggcctggtgggcggaggcaaagggggagccaggggcggagaaagggttgcccaagtctgggagtgagggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccagcaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctacttttttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctgcattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttcccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 33 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 34 - DNA, 3608 bp, Homo sapiens
gaattcgtgtctcggcactcactcccggccgcccggacagggagctttcgctggcgcgcttggccggcgacaggacaggttcgggacgtccatctgtccatccgtccggagagaaattacagatccgcagccccgggatggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcaatgtacctaatatataccaggacaccacaatttcttggtggaaagatgggaaggaattgcttgggggacatcatcgaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatcccccggcgtgctaactgttccaggcctgacggagatggcggtcttcagttgtgaggcccacaatgacaaagggctgaccgtgtcccagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgggtaatggctcagtcatgatttttaacacctctgccttaccacatctgtaccaaatcaagcagctgcaagccctggctaattacagcattggtgtttcctgcatgaatgaaataggctggtctgcagtgagcccttggattctagcaagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacatcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatacgtacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggccccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtgaggagaggtgcggggagacattccaaaaatcaagccaattcttctgctgtaggagaatccaattgtacctgatgtttttggtatttgtcttccttaccaagtgaactccatggccccaaagcaccagatgaatgttgttaaggaagctgtcattaaaaatacataatatatatttatttaaagagaaaaaatatgtgtatatcatgaaaaagacaaggatattttaataaaacattacttatttcatttcacttatcttgcatatcttaaaattaagcttcagctgctccttgatattaacctttgtacagagttgaagttgttttttcaacttcttttctttttcattactattaaatgtaaaaatatttgtaaaatgaaatgccatatttgacttggcttctggtcttgatgtatttgataagaatgattaattttctgatatggcttccataataaaattgaaatagga

SEQ ID NO: 35 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSINVPNIYQDTTISWWKDGKELLGGHHRITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPGVLTVPGLTEMAVFSCEAHNDKGLTVSQGVQINIKAIPSPPTEVSIRNSTAHSILISWVPGFDGYSPFRNCSIQVKEADPLGNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSHREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIRTRGMTPYPGVQNHEMYDYLLHGHRLKQPEDCLDELYEIMYSCWRTDPLDRPTFSVLRLQLEKLLESLPDVRNQADVIYVNTQLLESSEGLAQGPTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

(19) United States
(12) Patent Application Publication
(10) Pub. No.: US 2007/0142273 A1
(43) Pub. Date: Jun. 21, 2007

(54) Methods and compositions targeting tyrosine kinases for the diagnosis and treatment of osteoarthritis

(76) Inventors: Sherif Daouti, Hasbrouck Heights, NJ (US); Chandrika Saidapet Kumar, Acton, MA (US); Brian Jude Latario, Groton, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/553,730

(22) PCT Filed: Apr. 16, 2004
(86) PCT No.: PCT/EP04/04052

§ 371 Date: Oct. 14, 2005

Related U.S. Application Data
(60) Provisional application No. 60/463,935, filed on Apr. 18, 2003.

(51) Int. Cl. A61K 38/17; C12Q 1/68; G01N 33/567

(52) U.S. Cl. 514/12; 435/7.2; 435/6

(57) ABSTRACT

The invention discloses that members of a subfamily of receptor tyrosine kinases comprising TYRO3, Axl, cMer and ligands thereof, such as GAS6 are suitable targets for the development of new therapeutics to treat, prevent or ameliorate OA. The invention also relates to methods to treat and/or ameliorate OA and pharmaceutical compositions therefore comprising modulators with inhibitory effect on the expression or activity of members of this subfamily of receptor tyrosine and related ligands. The invention also relates to a method to identify compounds with therapeutic usefulness to treat OA, comprising identifying compounds that can, e.g., inhibit activity and/or expression of these polypeptides.

### BACKGROUND OF THE INVENTION

The present invention relates to methods and compositions for the diagnosis and treatment of osteoarthritis (OA). In particular, the invention discloses a subfamily of receptor tyrosine kinases which comprises TYRO3, Axl and cMer and the use of these genes, gene products and ligands thereto as suitable drug targets for the development of new therapeutic treatments for OA, and as diagnostic markers for OA. The invention also relates to screening assays for identifying compounds that are useful for treating OA and pharmaceutical compositions comprising said compounds.

### FIELD OF THE INVENTION

OA is primarily a non-inflammatory disease characterized by pain and stiffness of the joints caused by the progressive loss of articular cartilage. OA is among the most common age associated disease and is estimated to affect about 56 million individuals worldwide or 80% of the population greater than 60 years old. Although OA is generally considered a degenerative disorder, the disease is associated with activation of chondrocyte cells, the major cell type present in normal articular cartilage. Hallmarks of this cell activation include hypertrophy, proliferation, dedifferentiation, degradation of the existing extracellular matrix and, finally, apoptosis.

The molecular etiology of OA remains unknown. Current therapeutic methods for treating OA are therefore directed toward symptomatic relief such as reducing joint pain and secondary inflammatory changes rather than toward treating the disease's underlying causes. Pharmacological interventions that prevent disease progression are not currently available. Many patients thus progress to advanced stages of the disease where total joint replacement surgery is necessary. For a review, see Pritzker, in: Brandt et al., Eds., Osteoarthritis, Oxford University Press, pp. 50-61 (1998). See also, Sandell & Aigner, Arthritis Res., Vol. 3, No. 2, pp. 107-113 (2001).

Large scale sequencing of OA cDNA libraries has identified several putative gene products or “marker genes” that are expressed by diseased chondrocyte cells. See Stokes et al., Arhritis Rheum., Vol. 46, No. 2, pp. 404-419 (2002); Hu et al., J. Biol. Chem., Vol. 273, No. 51, pp. 34406-34412 (1998); Aigner et al., Arthritis Rheum., Vol. 44, No. 12, pp. 2777-2789 (2001); and Kumar et al., Osteoarthritis Cartilage., Vol. 9, No. 7, pp. 641-653 (2001). However, functional information is not presently available for these gene products and their role in OA, if any, remains unknown. The molecular basis of OA therefore remains unknown and only a very limited number of potential drug targets is known.

There remains a need, therefore, for therapeutic compounds and methods to treat OA and related diseases. There is moreover a need for novel genes and gene products that may be useful, e.g., as drug targets for such therapeutic methods to treat OA. There is also a need for novel methods, particularly novel screening assays, to identify such drug targets.

### SUMMARY OF THE INVENTION

The present invention relates to methods and compositions for the treatment and diagnosis of OA. In particular, the invention provides novel uses of members of the TYRO3 subfamily of receptor tyrosine kinases including, TYRO3, Axl and cMer and their ligands, e.g., GAS6 and PROS1. Applicants have surprisingly discovered that TYRO3 and its ligand, particularly GAS6, are expressed at elevated levels in cells and tissue from the cartilage of individuals who have OA. Moreover, elevated expression of TYRO3 or Axl in chondrocyte cells and/or the treatment of chondrocyte cells with a TYRO3 ligand induces the expression of several different genes associated with OA, whereas the suppression of TYRO3, Axl or cMer, e.g., by RNA interference, blocks IL-1 or TNF mediated induction of genetic markers of OA suggesting that the TYRO3 subfamily of receptor tyrosine kinases may have a key role in mediating cartilage degradation in vivo and as such are useful drug targets for the development of new therapeutics to treat, prevent or ameliorate OA.

Accordingly, the invention provides novel screening assays that use a member of the TYRO3 subfamily of receptor tyrosine kinases, e.g., TYRO3, AXL and cMer, and/or a ligand thereto, e.g., GAS6 or PROS1, to identify compounds for treating OA. In one aspect, these methods involve contacting a test compound to a reaction mixture that contains a TYRO3 subfamily member polypeptide and a ligand thereto, preferably under conditions that permit binding of the ligand to the polypeptide to form a binding complex. In a preferred aspect, the TYRO3 subfamily member polypeptide is TYRO3 and the ligand a TYRO3 ligand. Levels of formation of the binding complex can then be detected in the reaction mixture in the presence of the test compound, and these levels are compared to the level of binding complex formed in the absence of the test compound. In such methods, a decrease in the level of binding complex formed in the presence of the test compound indicates that the test compound can be used for treating OA.

In preferred embodiment of these methods, the TYRO3 polypeptide is a polypeptide comprising the amino acid sequence set forth in SEQ ID NO:2. In other preferred embodiments, the TYRO3 ligand is a GAS6 polypeptide, and preferably is a polypeptide that comprises the amino acid sequence set forth in SEQ ID NO:3.

In other aspects, the invention also provides diagnostic methods for identifying individuals who have OA. In one embodiment, these methods involve detecting a nucleic acid encoding a polypeptide member of the TYRO3 subfamily of receptor tyrosine kinases, preferably a TYRO3 nucleic acid, in a biological sample, e.g., in chondrocyte cells, cartilage tissue, and/or synovial fluid or serum, derived from an individual, e.g., a patient or other individual suspected of having OA, and comparing the level of said nucleic acid in said biological sample to levels of nucleic acid in individuals who do not have OA. In such embodiments, an elevated level of said nucleic acid in said biological sample from the individual indicates that the individual does have OA or, alternatively, that the individual has an increased risk of developing OA.

In other embodiments, diagnostic aspects of the invention involve detecting levels of a polypeptide belonging to the TYRO3 subfamily of receptor tyrosine kinases, preferably a TYRO3 polypeptide, in a biological sample, e.g., in chondrocyte cells, cartilage tissue and/or synovial fluid or serum derived from an individual, e.g., from a patient or other individual suspected of having OA, and comparing the level of said polypeptide in said biological sample to levels of said polypeptide in individuals who do not have OA. In such embodiments, an elevated level of said polypeptide in the biological sample from the individual indicates that the individual does have OA or, alternatively, that the individual has an increased risk of developing OA.

In particularly preferred embodiments of these diagnostic methods, the TYRO3 polypeptide may be a polypeptide having the amino acid sequence set forth in SEQ ID NO:2 and the TYRO3 nucleic acid may be a nucleic acid that encodes a polypeptide having the amino acid sequence set forth in SEQ ID NO:2.

Alternatively, the invention also provides diagnostic methods that use a ligand of a member of the TYRO3 subfamily of receptor tyrosine kinases, preferably a TYRO3 ligand, such as GAS6 or PROS1, to identify an individual having OA. For example, in one embodiment, such methods comprise detecting, in a biological sample, e.g., in chondrocyte cells, cartilage tissue and/or synovial fluid or serum derived from an individual, e.g., from a patient or other individual suspected of having OA, a nucleic acid that encodes a ligand to a member of the TYRO3 subfamily and comparing the level of nucleic acid in said biological sample to levels of the nucleic acid in individuals who do not have OA. In such embodiment, an elevated level of the nucleic acid encoding said ligand indicates that the individual does have OA or, alternatively, that the individual has an increased risk of developing OA.

In other embodiments, diagnostic aspects of the invention involve detecting a TYRO3 subfamily member ligand polypeptide, preferably a TYRO3 ligand polypeptide, in a biological sample, e.g., in chondrocyte cells, cartilage tissue and/or synovial fluid or serum derived from an individual, e.g., from a patient or other individual suspected of having OA, and comparing the level of said ligand polypeptide in said biological sample to levels of the ligand polypeptide in individuals who do not have OA. In such embodiment, an elevated level of said ligand polypeptide in said biological sample from the individual indicates that the individual does have OA or, alternatively, that the individual has an increased risk of developing OA.

In particularly preferred embodiments of these diagnostic methods, the TYRO3 ligand is preferably a GAS6 polypeptide, such as a polypeptide comprising the amino acid sequence set forth in SEQ ID NO:3. Likewise, the nucleic acid encoding the TYRO3 ligand is preferably a nucleic acid encoding GAS6, such as a nucleic acid that encodes the amino acid sequence set forth in SEQ ID NO:3. In a particularly preferred embodiment, the nucleic acid encoding a TYRO3 ligand comprises the nucleotide sequence set forth In SEQ ID NO:4.

In another aspect, the invention relates to a method to treat, prevent or ameliorate OA, comprising administering to a subject in need thereof a pharmaceutical composition comprising an effective amount of a modulator of a TYRO3 subfamily member and/or ligand thereof. In various preferred embodiments, said pharmaceutical composition comprises one or more modulators to TYRO3, Axl, cMer and/or ligands thereof.

In another aspect, the invention relates to a pharmaceutical composition comprising a modulator of a TYRO3 subfamily member and/or ligand thereof in an amount effective to treat, prevent or ameliorate OA in a subject in need thereof wherein said modulator, e.g., can inhibit the activity, expression of or ligand binding to, any one or more of the members of this subfamily, e.g., TYRO3, Axl and cMer. In one embodiment, said pharmaceutical composition comprises any one or more substances selected from the group consisting of antisense oligonucleotides, triple helix DNA, siRNA, ribozymes, RNA aptamers or double- or single-stranded RNA directed to a nucleic acid sequence of a TYRO3 subfamily member or ligand thereof wherein said substances are designed to inhibit expression of said family member or ligand. In a further embodiment, said pharmaceutical composition comprises antibodies to a TYRO3 subfamily member or ligand thereof, or fragments thereof, wherein said antibodies can, e.g., inhibit the activity of said member and/or ligand.

In yet another aspect of the present invention there are provided assay methods and kits comprising the components necessary to detect expression of polynucleotides encoding a TYRO3 subfamily member or ligand thereof, or polypeptide levels of said members or ligands thereof, or fragments thereof, in biological samples derived from a patient, such kits comprising, e.g., antibodies that bind to said polypeptides, or to fragments thereof or oligonucleotide probes that hybridize with said polynucleotides. In a preferred embodiment, such kits also comprise instructions detailing the procedures by which the kit components are to be used.

### DETAILED DESCRIPTION OF THE INVENTION

The present invention relates to a subfamily of receptor tyrosine kinases referred to herein as the TYRO3 subfamily of receptor tyrosine kinases which comprises the homologous proteins, TYRO3, Axl and cMer. Data disclosed herein indicate that these polypeptides are involved in the pathogenesis of OA and as such it is contemplated herein that these polypeptides and ligands thereof are suitable drug targets for the development of therapeutics to treat, prevent or ameliorate OA and related conditions.

The TYRO3 polypeptide has been described, e.g., by Polyi et al., Gene, Vol. 134, No. 2, pp. 289-293 (1993); and Schultz et al., Mol. Brain. Res., Vol. 28, pp. 273-280 (1995). TYRO3 is also known as SKY, TIF, RSE, DTK and BRT. See Ohashi et al., Oncogene, Vol. 9, pp. 699-705 (1994); Dai et al., Oncogene, Vol. 9, pp. 975-979 (1994); Mark et al., J. Biol. Chem., Vol. 269, pp. 10720-10728 (1994); and Fujimoto et al., Oncogene, Vol. 9, pp. 693-698 (1994). A preferred TYRO3 amino acid sequence is available from the GenBank database and has the Accession No. NP_{—}006284 (SEQ ID NO:2). A preferred cDNA sequence that encodes this TYRO3 polypeptide is also available from GenBank and has the Accession No. NM_{—}006293 (SEQ ID NO:1). Nucleotides 225-2897 of that cDNA sequence correspond to an open reading frame (ORF) or “coding sequence” (CDS) that encodes the amino acid sequence of SEQ ID NO:2.

The TYRO3 receptor has structural and sequence homology with the receptor tyrosine kinase Axl also known as UFO or ARK (for a description, see O'Bryan et al., Mol. Cell. Biol., Vol. 11, pp. 5016-5013 (1991); Janssen et al., Oncogene, Vol. 6, pp. 2113-2120 (1991); and Rescigno et al., Oncogene, Vol. 6, pp. 1909-1913 (1991); GenBank Accession Numbers of Axl splice variants Axlv1 and Axlv2, Gen Bank Accession No. NM_{—}021913; SEQ ID NO:32 and GenBank Accession No. NM_{—}001699, SEQ ID NO:30, respectively); and with the receptor tyrosine kinase cMer also known as EYK (see Graham et al., Cell Growth Differ., Vol. 5, pp. 647-657 (1994); Jia et al., J. Biol. Chem., Vol. 269, pp. 1839-1844 (1994); GenBank Accession Numbers NM_{—}006343 and NP_{—}006334; and SEQ ID NO:34 and SEQ ID NO:35). Together, these three polypeptides define a sub-family of tyrosine kinase receptors that each have similar ectodomains, sharing about 35% amino acid sequence identity and comprising two immunoglobulin-like domains and two fibronectin type III repeats. See Schulz et al., Mol. Brain. Res., Vol. 28, pp. 273-280 (1995).

At least one ligand has been described that specifically binds to and activates TYRO3. See, e.g., Varnum et al., Nature, Vol. 373, pp. 623-626 (1995); Stitt et al., Cell, Vol. 80, No. 4, pp. 661-670 (1995); and Manfioletti et al., Mol. Cell. Biol., Vol. 13, pp. 4976-4985 (1993). An exemplary amino acid sequence for this ligand, which is known as “growth arrest-specific protein 6” or “GAS6” is available from the GenBank database and has the Accession No. NP_{—}000811 (SEQ ID NO:3). A preferred nucleotide sequence encoding that GAS6 polypeptide is available from the GenBank Accession No. NM_{—}000820 (SEQ ID NO:4). In particular, this cDNA sequence contains an open reading frame comprising nucleotide residues 135-2171 of SEQ ID NO:4. GAS6 also has been shown to be a ligand for other TYRO3 subfamily members, Axl and cMer. See, e.g., Nagata et al., J. Biol. Chem., Vol. 271, pp. 30022-30027 (1996).

Another ligand that specifically binds to TYRO3 is a protein termed Protein S. See Stitt et al. (1995), supra; and Wimmel et al., Cancer, Vol. 86, No. 1, pp. 4349 (1999). A preferred nucleotide sequence encoding that Protein S polypeptide (PROS1) is available from the GenBank Accession No. NM_{—}000313 and is also provided here at SEQ ID NO:28). An exemplary, preferred PROS1 amino acid sequence is also available from the GenBank Accession No. NP_{—}000304 (SEQ ID NO:29).

As disclosed in the examples below, Applicants have discovered that TYRO3, Axl, cMer and GAS6 are implicated in the pathogenesis of OA. For example, data indicates that expression of the TYRO3 gene in chondrocyte cells induces several genetic markers that are associated with OA including Aggrecanase-1, MMP-13, iNOS and COX-2-, while inhibition of TYRO3 in chondrocyte cells reduces the expression of these marker genes. Applicants have also discovered that both TYRO3 and GAS6 are expressed at elevated levels in chondrocyte cells from OA patients compared to the expression of this gene seen in normal chondrocyte cells. Moreover, treatment of chondrocyte cells with GAS6 also induces OA marker genes. In addition, experiments with siRNA also support a role for cMer and Axl splice variants in the pathogenesis of OA.

These surprising discoveries demonstrate that these receptor tyrosine kinases, as well as ligands thereto, e.g., GAS6 and PROS1, may play an important role in mediating OA, as well as other cartilage disorders. In particular, these findings demonstrate that these receptor tyrosine kinases, as well as ligands thereto may be used, e.g., as drug targets for the development of therapeutics to treat, prevent or ameliorate OA (also referred to herein as “treating” OA for simplicity).

Accordingly, the present invention provides screening assays by which drugs and other therapeutic compounds for treating OA may be identified. In particular, the screening assays of the invention include those that identify compounds which specifically bind to a gene encoding a member of the TYRO3 subfamily, preferably to the TYRO3 gene or gene product and inhibit its expression or activity. Alternatively, the invention also provides screening assays that identify compounds which bind to a gene or gene product of a TYRO3 subfamily specific ligand, e.g., GAS6, and inhibit the ligand's expression or activity. In addition, the invention also provides screening assays that identify compounds which inhibit or otherwise modulate the binding of a TYRO3 receptor to a TYRO3 ligand, e.g., to GAS6.

The compounds and modulators identified in such assays can themselves be used, e.g., in pharmaceutical compositions and/or therapeutic methods for treating OA. Such pharmaceutical compositions and therapeutic methods are therefore provided, infra, and are also part of the present invention.

Finally, Applicants' discoveries also demonstrate that members of the TYRO3 subfamily of polypeptides disclosed herein and respective ligand(s) thereto can be used in prognositc and diagnostic methods, to identify osteoarthritic cells and/or to identify individuals, e.g., patients, who have OA. Examples of such diagnostic and prognostic methods are provided below, and are also part of the present invention.

The present invention and its description may employ a variety of conventional techniques in the arts of molecular biology, microbiology and recombinant DNA technology. Such techniques are well-known in the art and are explained fully in the literature. See, e.g., Sambrook, Fitsch & Maniatis, Molecular Cloning: A Laboratory Manual, 2^{nd} Edition, Cold Spring Harbor Laboratory Press, Cold Spring Harbor, N.Y. (1989), referred to herein as “Sambrook et al. (1989)”; D. N. Glover et al., DNA Cloning: A Practical Approach, Vols. I and II (1985); Gait, Ed., Oligonucleotide Synthesis (1984); Hames & S. J. Higgins, Eds., Nucleic Acid Hybridization (1984); Freshney, Ed., Animal Cell Culture (1986); Immobilized Cells and Enzymes, IRL Press (1986); Perbal, A Practical Guide to Molecular Cloning (1984); and Ausubel et al., Eds., Current Protocols in Molecular Biology, John Wiley & Sons, Inc.

TYRO3 Polypeptides

The present invention relates to a subfamily of tyrosine kinases which include TYRO3, Axl, cMer and variants thereof. More specifically, the present invention relates to the polypeptide TYRO3 that is described, e.g., by Polyi et al., Gene, Vol. 134, No. 2, pp. 289-293 (1993). In particular, the present invention provides uses of TYRO3, cMer and Axl in medicaments and pharmaceutical compositions to treat OA and other cartilage disorders. Accordingly, the invention provides methods and compositions that use TYRO3 subfamily members to diagnose and/or treat OA and other cartilage disorders.

In one specific embodiment, a TYRO3 polypeptide is derived from a human cell and/or has the amino acid sequence of a TYRO3 polypeptide derived from a human cell. For example, a particularly preferred TYRO3 polypeptide may comprise the amino acid sequence set forth in GenBank Accession No. NP_{—}006284 (SEQ ID NO:2). However, it is understood that the TYRO3 polypeptides are not limited to this particular sequence, but also include homologs and variants familiar to one of ordinary skill in the art.

Hence, TYRO3 polypeptides also include polypeptides comprising an amino acid sequence for one or more epitopes or domains of a full length TYRO3 polypeptide. An epitope of a polypeptide represents a site on the polypeptide against which an antibody may be produced and to which the antibody binds. Therefore, polypeptides comprising the amino acid sequence of a TYRO3 epitope are useful for making antibodies to the TYRO3 polypeptide. Preferably, an epitope comprises a sequence of at least 5, more preferably at least 10, 15, 20, 25 or 50 amino acid residues in length. Thus, polypeptides that comprise epitopes of a TYRO3 preferably contain an amino acid sequence corresponding to at least 5, at least 10, at least 15, at least 20, at least 25 or at least 50 amino acid residues of a full-length TYRO3 polypeptide sequence.

TYRO3 polypeptides also include analogs and derivatives of the exemplary full-length TYRO3 polypeptide sequence provided in SEQ ID NO:2. Analogs and derivatives of the TYRO3 polypeptides have the same or homologous characteristics of the exemplary TYRO3 polypeptide set forth in SEQ ID NO:2. Chimeric or fusion polypeptides can also be prepared in which the TYRO3 portion of the fusion polypeptide has one or more characteristics of a TYRO3 polypeptide. Such fusion polypeptides therefore represent embodiments of TYRO3 polypeptides. Such fusion polypeptides may also comprise the amino acid sequence of a marker polypeptide, e.g., FLAG, a histidine tag, glutathione S-transferase (GST), or the Fc portion of an IgG to name a few. Additionally, fusion polypeptides may comprise amino acid sequences that increase solubility of the polypeptide, such as a thioreductase amino acid sequence or the sequence of one or more immunoglobulin proteins, e.g., IgG1 or IgG2.

Analogs or variants of a TYRO3 subfamily member polypeptide can also be made by altering encoding nucleic acid molecules, for example by substitutions, additions or deletions. Specifically, preferred analogs or variants of a TYRO3 polypeptide include “function conservative variants” of the particular TYRO3 polypeptide sequence specified in SEQ ID NO:2. “Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. Such changes are expected to have little or no effect on the apparent molecular weight or isoelectric point of the polypeptide. Hence, such altered nucleic acid molecules preferably encode functionally similar molecules, i.e., molecules that perform one or more functions of a TYRO3 polypeptide and/or have one or more of the TYRO3 polypeptide's bioactivities.

Amino acid residues, other than ones that are specifically identified herein as being conserved, may differ among variants of a protein or polypeptide. Accordingly, the percentage of protein or amino acid sequence similarity between any two variants or analogs of a polypeptide member of the TYRO3 subfamily may vary. Typically, the percentage of protein or amino acid sequence similarity between variant or analogs of these polypeptides may be from 70-99%, as determined according to an alignment scheme, such as the Cluster Method and/or the MEGALIGN or GCG alignment algorithm. Preferred variants and analogs of these polypeptide are at least about 75%, and more preferably at least about 80%, 85%, 90%, 95% or 99% sequence identity as determined by a sequence comparison algorithm, such as BLAST, FASTA, DNA Strider, CLUSTAL, etc.

Function-conservative variants, as defined above, include not only variants of the full length TYRO3 subfamily polypeptides discussed herein including, e.g., variants of polypeptides comprising the particular TYRO3 subfamily polypeptide sequences specified in the Examples, infra, but also include function-conservative variants of modified TYRO3 subfamily polypeptides, e.g., truncations and deletions; and of fragments, e.g., corresponding to domains or epitopes, of full-length TYRO3 subfamily polypeptides.

In yet other embodiments, an analog of a TYRO3 polypeptide is an allelic variant or mutant of a TYRO3 polypeptide sequence provided in SEQ ID NO:2. The terms allelic variant and mutant, when used herein to describe a polypeptide, refer to a polypeptide encoded by an allelic variant or mutant gene. Thus, the allelic variant and mutant TYRO3 polypeptides are polypeptides encoded by allelic variants or mutants of a TYRO3 nucleic acid.

In yet other embodiments, an analog of a TYRO3 subfamily member polypeptide is a substantially homologous polypeptide from the same species, e.g., allelic variants; or from another species, e.g., an orthologous polypeptide. The term “homologous,” in all its grammatical forms and spelling variations, refers to the relationship between two proteins or nucleic acids that possess a “common evolutionary origin”, including proteins from superfamilies, e.g., the immunoglobulin superfamily, in the same species of organism, as well as homologous proteins from different species of organism, e.g., myosin light chain polypeptide, etc. See Reeck et al., Cell, Vol. 50, p. 667 (1987). Such proteins (and their encoding nucleic acids) have sequence homology, as reflected by their sequence similarity, whether in terms of percent identity or by the presence of specific residues or motifs and conserved positions. Preferred homologous polypeptides of the present invention have levels of sequence similarity or identity as specified, above, for other variant and analog TYRO3 subfamily member polypeptides of the invention. Homologs and orthologs of these polypeptides may be obtained, e.g., from mammals, such as humans, mice, rats, hamsters, rabbit, guinea pig, dog, cat, sheep, goat, pig, horse and cow, to name a few.

In other embodiments, variants of a TYRO3 polypeptide, including analogs, homologs, etc., are polypeptides encoded by nucleic acid molecules that hybridize to the complement of a nucleic acid molecule encoding one or more of the particular TYRO3 polypeptide sequence set forth in SEQ ID NO:2. A nucleic acid molecule is “hybridizable” to another nucleic acid molecule, e.g., cDNA, genomic DNA or RNA, when a single-stranded form of the nucleic acid molecule can anneal to the other nucleic acid molecule under appropriate conditions of temperature and solution ionic strength. See, e.g., Sambrook et al., supra. The conditions of temperature and ionic strength determine the “stringency” of the hybridization. For preliminary screening for homologous nucleic acids, low stringency hybridization conditions corresponding to a melting temperature of about 55° C. can be used, e.g., 5×SSC, 0.1% SDS, 0.25% milk and no formamide; or, alternatively, 30% formamide, 5×SSC and 0.5% SDS. Moderate stringency hybridization conditions correspond to a higher Tₘ, e.g., 40% formamide with 5× or 6×SSC. High-stringency hybridization conditions correspond to the highest Tₘ, e.g., 50% formamide, 5× or 6×SSC. A 1×SSC solution is understood to be a solution containing 0.15 M NaCl and 0.015 M Na-citrate.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well-known in the art. The greater the degree of similarity or homology between two nucleotide sequences the greater the value of Tₘ for hybrids of nucleic acids having those sequences.

For hybrids of greater than 100 nucleotides in length, equations for calculating Tₘ have been derived. See Sambrook et al., supra, pp. 9.50-9.51.

In a specific embodiment, the term “standard hybridization conditions” refers to a Tₘ of about 55° C. and utilizes conditions as set forth above. In a preferred embodiment, the Tₘ is 60° C.; in a more preferred embodiment, the Tₘ is 65° C. In a specific embodiment, the term “high-stringency” refers to hybridization and/or washing conditions at 68° C. in 0.2×SSC, at 42° C. in 50% formamide, 4×SSC, or under conditions that afford levels of hybridization equivalent to those observed under either of these two conditions.

In still other embodiments, variants (including analogs, homologs and orthologs) of a TYRO3 subfamily member polypeptide can be identified by isolating variants of a TYRO3 subfamily member gene, e.g., using PCR with degenerate oligonucleotide primers designed on the basis of amino acid sequences of the TYRO3 subfamily polypeptides and as described below.

Derivatives of a TYRO3 subfamily polypeptide further include phosphorylated polypeptides, myristylated polypeptides, methylated polypeptides and other polypeptides that are chemically modified. TYRO3 subfamily polypeptides further include labeled variants, e.g., radio-labeled with iodine or phosphorous (see, e.g., EP 372707B) or other detectable molecules, such as but by no means limited to, biotin, fluorescent dyes, e.g., Cy5 or Cy3, a chelating group complexed with a metal ion, a chromophore or fluorophore, a gold colloid, a particle, such as a latex bead, or attached to a water soluble polymer, such as poly(ethylene)glycol (PEG). Chemical modifications of a TYRO3 subfamily polypeptide may provide additional advantages under certain circumstances. See, e.g., U.S. Pat. No. 4,179,337. For a review, see also Abuchowski et al., Enzymes as Drugs, Holcerberg & Roberts, Eds., pp. 367-383 (1981). A review article describing protein modification and fusion proteins is also found in Fracis, Focus on Growth Factors, Vol. 3, pp. 4-10, Mediscript: Mountview Court, Friem Bamet Lane, London N20, OLD, UK (1992).

TYRO3 Subfamily Member Nucleic Acids

In general, a nucleic acid encoding a polypeptide member of the TYRO3 subfamily of tyrosine kinases comprises a nucleic acid sequence that encodes a complement of a nucleic acid sequence that encodes a TYRO3 subfamily member polypeptide, and fragments thereof. Thus, in one preferred embodiment a TYRO3 nucleic acid molecule comprises a nucleotide sequences that encodes the amino acid sequence set forth in SEQ ID NO:2. In a particularly preferred aspect of this embodiment the TYRO3 nucleic acid molecule has a nucleotide sequence that comprises the coding portion, i.e., the ORF of the nucleotide sequence set forth in GenBank Accession No. NM_{—}006293 and provided here at SEQ ID NO:1. A particularly preferred nucleic acid molecule comprises the sequence of nucleotides 225-2897 of the nucleotide sequence set forth in SEQ ID NO:1. Similarly, Axl kinase polypeptide variants AXLv1 and AXLv2 have nucleotide sequences as provided herein at SEQ ID NOs.30 and 32, respectively, and cMer at SEQ ID NO.34.

In still other embodiments, the TYRO3 subfamily nucleic acid molecules comprise nucleotide sequences that encode one or more key domains of a TYRO3 subfamily member polypeptide including, e.g., receptor binding sites, kinase, extracellular and transmembrane domains.

The TYRO3 subfamily nucleic acid molecules also include nucleic acids which comprise a sequence encoding one or more fragments of a TYRO3 subfamily polypeptide sequence.

The TYRO3 subfamily nucleic acid molecules also include nucleic acid molecules that comprise coding sequences for modified polypeptides, e.g., having amino acid substitutions, deletions or truncations; and for variant, including allelic variants, analogs and homologs from the same or different species, polypeptides. In preferred embodiments, such nucleic acid molecules have at least 50%, preferably at least 75% and more preferably at least 90% sequence identity to a TYRO3 subfamily polypeptide coding sequence, e.g., to the coding sequences set forth in SEQ ID NO:1, SEQ ID NO:30, SEQ ID NO:32 or SEQ ID NO:34.

In addition, nucleic acid molecules encoding TYRO3 subfamily member polypeptides include those that hybridize to another nucleic acid molecule, e.g., in a Southern blot assay under defined conditions. For example, in specific embodiments a TYRO3 nucleic acid molecule comprises a nucleotide sequence which hybridizes to a complement of a particular nucleic acid sequence, such as the TYRO3 coding sequence set forth in SEQ ID NO:1. Alternatively, a nucleic acid molecule may hybridize, under the same defined hybridization conditions, to the complement of a fragment of a nucleotide sequence encoding a full-length TYRO3 polypeptide. Examples of preferred hybridization include those set forth above.

In other embodiments, the nucleic acid molecules comprise fragments of a full-length TYRO3 subfamily member nucleic acid sequence. Such nucleic acid fragments comprise a nucleotide sequence that corresponds to a sequence of at least 10 nucleotides, preferably at least 15 nucleotides and more preferably at least 20 nucleotides of a nucleotide sequence encoding a full-length TYRO3 subfamily member polypeptide. In preferred embodiments, the nucleic acid fragments comprise sequences of at least 10, preferably at least 15 and, more preferably, at least 20 nucleotides that are complementary and/or hybridize to a full-length TYRO3 subfamily member nucleic acid sequence or to a fragment thereof. For hybridization with shorter nucleic acids, i.e., oligonucleotides, the position of mismatches becomes more important and the length of the oligonucleotide determines its specificity. See Sambrook et al., supra, pp. 11.7-11.8. A minimum length for a hybridizable nucleic acid is preferably at least about 10 nucleotides, more preferably at least about 15 nucleotides, and still more preferably at least about 20 nucleotides.

Nucleic acid molecules comprising such fragments are useful, e.g., as oligonucletide probes and primers, e.g., PCR primers, to detect and amplify other nucleic acid molecules encoding a TYRO3 subfamily member polypeptide, including genes the encode variant polypeptides. Oligonucleotide fragments may also be used, e.g., as antisense nucleic adds to modulate levels of a TYRO3 subfamily member gene's expression or transcription in cells.

The nucleic acid molecules also include “chimeric” nucleic acid molecules. Such chimeric nucleic acid molecules are polynucleotides which comprise at least one TYRO3 subfamily member nucleic acid sequence, e.g., which may be any of the full-length or partial TYRO3, AXL or cMer nucleic acid sequences described above, and also at least one non-TYRO3 subfamily member nucleic acid sequence, i.e., a nucleic acid sequence not normally associated with the naturally-occurring subfamily member gene. For example, the non-subfamily member nucleic acid sequence may be a heterologous regulatory sequence, e.g., a promoter sequence, that is derived from another gene and is not normally associated with the naturally-occurring subfamily member gene. The non-subfamily member nucleic acid sequence may also be a coding sequence of another polypeptide, such as FLAG, a histidine tag, glutathione S-transferase (GST), hemaglutinin, β-galactosidase, thioreductase or an immunoglobulin domain or domains, e.g., an Fc region. In preferred embodiments, a chimeric nucleic acid molecule encodes a fusion polypeptide of the invention.

Nucleic acid molecules, whether genomic DNA, cDNA or otherwise, can be isolated from any source including, e.g., cDNA or genomic libraries derived from a cell or cell line from an organism that has the desired TYRO3 subfamily member gene. In the case of cDNA libraries, such libraries are preferably derived from a cell or cell line that expresses the particular TYRO3 subfamily member gene. Methods for obtaining genes are well-known in the art. See, e.g., Sambrook et al. (1989), supra.

The DNA may be obtained by standard procedures known in the art from cloned DNA, e.g., from a DNA “library”, and preferably is obtained from a cDNA library prepared from tissues with high level expression of the protein. In one preferred embodiment, the DNA is obtained from a “subtraction” library to enrich the library for cDNAs of genes specifically expressed by a particular cell type or under certain conditions. Use of such a subtraction library may increase the likelihood of isolating cDNA for a particular gene. In still other embodiments, a library may be prepared by chemical synthesis, by cDNA cloning or by the cloning of genomic DNA or fragments thereof purified from the desired cell. See, e.g., Sambrook et al. (1989), supra; Glover, Ed., DNA Cloning: A Practical Approach, MRL Press, Ltd. Oxford, U.K., Vols. I and II (1985).

In one embodiment, a cDNA library may be screened for a desired TYRO3 subfamily member nucleic acid by identifying cDNA inserts that encode a polypeptide which is homologous or substantially similar to a TYRO3 subfamily member polypeptide, e.g., the TYRO3 polypeptide set forth in SEQ ID NO:2. Similarly, a cDNA library may be screened for a desired TYRO3 subfamily member nucleic acid by identifying cDNA inserts having a nucleic acid sequence that is homologous or substantially similar to a TYRO3 subfamily member nucleotide sequence.

Clones derived from genomic DNA may contain regulatory and intron DNA regions in addition to coding regions. Clones derived from cDNA generally will not contain intron sequences. Whatever the source, the gene is preferably molecularly cloned into a suitable vector for propagation of the gene. Identification of the specific DNA fragment containing the desired TYRO3 subfamily member gene may be accomplished in a number of ways. For example, a portion of a TYRO3 subfamily member gene can be purified and labeled to prepare a labeled probe. See Benton & Davis, Science, Vol. 196, p. 180 (1977); and Grunstein & Hogness, Proc. Natl. Acad. Sci. USA, Vol. 72, p. 3961 (1975). Those DNA fragments with substantial homology to the probe, such as an allelic variant from another individual, will hybridize. In a specific embodiment, highest-stringency hybridization conditions are used to identify a homologous TYRO3 subfamily member gene.

The genes encoding derivatives and analogs of a TYRO3 subfamily member gene of interest can be produced by various methods known in the art. The manipulations which result in their production can occur at the gene or protein level. For example, the cloned sequence can be modified by any of numerous strategies known in the art. See Sambrook et al. (1989), supra. The sequence can be cleaved at appropriate sites with restriction endonuclease(s), followed by further enzymatic modification if desired, isolated and ligated in vitro. In the production of the gene encoding a derivative or analog of a desired gene, care should be taken to ensure that the modified gene remains within the same translational reading frame as the gene from which it is derived, uninterrupted by translational stop signals, in the gene region where the desired activity is encoded.

Additionally, the nucleic acid sequence encoding the TYRO3 subfamily member can be mutated in vitro or in vivo, to create and/or destroy translation, initiation and/or termination sequences, or to create variations in coding regions and/or form new restriction endonuclease sites or destroy preexisting ones, to facilitate further in vitro modification. Modifications can also be made to introduce restriction sites and facilitate cloning the gene into an expression vector. Any technique for mutagenesis known in the art can be used including, but not limited to, in vitro site-directed mutagenesis (see Hutchinson et al., J. Biol. Chem., Vol. 253, p. 6551 (1978); Zoller and Smith, DNA, Vol. 3, pp. 479-488 (1984); Oliphant et al., Gene, Vol. 44, p. 177 (1986); and Hutchinson et al., Proc. Natl. Acad. Sci. USA, Vol. 83, p. 710 (1986); use of TAB linkers. See Pharmacia Corp., Peapack, N.J., etc. PCR techniques are preferred for site directed mutagenesis. See Higuchi, PCR Technology: Principles and Applications for DNA Amplification, Erlich, Ed., Stockton Press, Chapter 6, pp. 61-70 (1989).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages, such as lambda derivatives, or plasmids, such as pBR322 derivatives or pUC plasmid derivatives, e.g., pGEX vectors, pmal-c, pFLAG, pKK plasmids (Clonetech, Palo Alto, Calif.), pET plasmids (Novagen, Inc., Madison, Wis.), pRSET or pREP plasmids, pcDNA (Invitrogen, Carlsbad, Calif.) or pMAL plasmids (New England Biolabs, Beverly, Mass.), etc. The insertion into a cloning vector can, e.g., be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini. These ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, If such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2m plasmid.

It is understood that the nucleic acids encoding the TYRO3 subfamily members disclosed herein may be either DNA or RNA and may be single-, double- or even triple-stranded, e.g., a triple-helix of TYRO3 single-stranded TYRO3 nucleic acids and/or their complement(s). TYRO3 subfamily member nucleic acids include genomic DNA, cDNA, RNA, mRNA, cRNA, etc.; as well as synthetic and genetically manipulated polynucleotides and both sense and antisense polynucleotides. Such synthetic polynucleotides Include, e.g., “protein nucleic acids” (PNA) formed by conjugating nucleotide bases to an amino acid backbone. Other exemplary synthetic nucleic acids include nucleic acids containing modified bases, such as thio-uracil, thio-guanine and fluoro-uracil. For convenience, the exemplary nucleotide sequences provided in this description are provided as sequences of DNA. However, it is understood that identical sequences of other types of nucleic acids, e.g., RNA, may also be used and are equivalent. Thus, e.g., where the particular nucleotide sequences in this description specify a thymine (T) at some position, it is understood that a uracil (U) may be substituted at that position and is a functional equivalent. Other equivalent substitutions will also be apparent to those of ordinary skill in the art and are therefore encompassed by the TYRO3 subfamily member nucleic acids disclosed herein.

The polynucleotides may be flanked by natural regulatory sequences, or they may be associated with heterologous sequences, such as promoters, enhancers, response elements, signal sequences, polyadenylation sequences, introns, 5′ and 3′-non-coding regions and the like. The term “heterologous”, in this context, refers to a combination of elements, e.g., sequences, that are not naturally-occurring. Hence, a TYRO3 subfamily member nucleic acid may have sequences, such as a promoter etc., that are not normally associated with the TYRO3 subfamily member gene.

Nucleic acids may also be modified by any means known in the art. Non-limiting examples of such modifications include methylation, “caps”, substitution of one or more of the naturally-occurring nucleotides with an analog, and internucleotide modifications, such as, e.g., those with uncharged linkages, e.g., methyl phosphonates, phosphotriesters, phosphoroamidites, carbamates, etc.; and with charged linkages, e.g., phosphorothioates, phosphorodithioates, etc. Nucleic acids may contain one or more additional covalently-linked moieties, such as proteins, e.g., nucleases, toxins, antibodies, signal peptides, poly-L-lysine, etc.; intercalators, e.g., acridine, psoralen, etc.; chelators, e.g., metals, radioactive metals, iron, oxidative metals, etc.; and alkylators, to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like.

TYRO3 Ligand Polypeptides and Nucleic Acids:

The present invention also relates to ligands of polypeptides belonging to the TYRO3 subfamily of receptor tyrosine kinases disclosed herein. Accordingly, said ligand polypeptides and nucleic acids are also provided and are considered a useful aspect of the present invention.

A “ligand” is, broadly speaking, any molecule that binds to another molecule. In preferred embodiments, the ligand is either a soluble molecule, is the smaller of the two binding molecules, or both. The other molecule is referred to as a “receptor.” In preferred embodiments, both a ligand and its receptor are molecules (preferably polypeptides) produced by cells. Preferably a ligand is a soluble molecule and the receptor is an integral membrane protein, i.e., a protein expressed on the surface of a cell.

Preferably, a ligand “specifically binds” to its receptor and/or vice-versa. The term “specific binding” refers to the ability of a ligand to distinguish between its receptor and other substances, e.g., other molecules, under physiological conditions. It is preferred that a ligand bind to its receptor with some affinity and vice-versa. Typically, the binding affinity of a ligand to its receptor is defined by a dissociation constant K_{d}. The dissociation constant K_{d} preferably has a value that is less than about 10 μM, is more preferably less than about 1 μM, and is still more preferably less than about 100 nM. In particularly preferred embodiments, the dissociation constant K_{d} has a value that is less than about 10 nM.

The binding of a ligand to its receptor is frequently a step of signal transduction within a cell. Non-limiting examples of ligand-receptor interactions include, but are not limited to, binding of a hormone to a hormone receptor, e.g., the binding of estrogen to the estrogen receptor, and the binding of a neurotransmitter to a receptor on the surface of a neuron. In this case, a TYRO3 ligand will stimulate tyrosine kinase activity upon binding to a TYRO3 receptor. Assays for tyrosine kinase activity are well known in the art and can be used to identify a ligand.

A particularly preferred ligand that specifically binds to and activates TYRO3 is described, e.g., by Varnum et al. (1995), supra; see also, Stitt et al. (1995), supra; and Manfioletti et al. (1993), supra. An exemplary amino acid sequence for this ligand, which is commonly referred to as “growth arrest-specific protein 6” or “GAS6” is set forth in GenBank under the Accession No. NP_{—}000811 and is also provided here in SEQ ID NO:3. This amino acid sequence is encoded by an open reading frame comprising residues 135-2171 of the full-length cDNA sequence set forth in GenBank Accession No. NM_{—}000820 and provided here at SEQ ID NO:4.

Accordingly, preferred TYRO3 subfamily ligand polypeptides include GAS6 polypeptides, such as the GAS6 polypeptide whose amino acid sequence is set forth in SEQ ID NO:3. The invention provides assays, described infra, by which those skilled in the art may identify still other TYRO3 ligand polypeptides and nucleic acids encoding such TYRO3 ligand polypeptides. The TYRO3 subfamily ligand polypeptides also encompass variant TYRO3 subfamily ligand polypeptides, including variant GAS6 polypeptides. These include variants, such as homologs, orthologs, derivatives, mutants, chimerics, fusions, fragments, truncated forms, etc. of a TYRO3 subfamily ligand polypeptide. Such TYRO3 subfamily ligand variants are defined as provided, supra, for TYRO3 subfamily receptor polypeptides.

Similarly, the invention also provides TYRO3 subfamily ligand nucleic acids, including nucleic acids that encode the GAS6 polypeptide of SEQ ID NO:3, e.g., nucleic acids comprising the sequence of nucleotides 135-2171 in SEQ ID NO:4; as suitable drug targets for OA as disclosed herein. Still other TYRO3 subfamily ligand nucleic acids may be identified using the screening assays described, infra, and such TYRO3 subfamily ligand nucleic acids may be used according to the methods of the present invention. The TYRO3 subfamily ligand nucleic acids also encompass variant TYRO3 subfamily ligand nucleic adds, including variant GAS6 nucleic acids. These include variants, such as homologs, orthologs, derivatives, mutants, chimerics, fusions, fragments, truncated forms, etc. of a TYRO3 subfamily ligand nucleic acid. Such variant TYRO3 subfamily ligand nucleic acids are defined as provided, supra, for TYRO3 subfamily receptor nucleic acids.

Expression of TYRO3 Subfamily Members and Ligands Thereto:

Nucleotide sequences encoding polypeptide members of the TYRO3 subfamily of tyrosine kinases or ligands thereto including chimeric proteins, antigenic fragments, derivatives or analogs thereof, may be inserted into an appropriate expression vector, i.e., a vector which contains the necessary elements for the transcription and translation of the inserted protein-coding sequence. Thus, e.g., a nucleic acid encoding a TYRO3 subfamily receptor or ligand can be operationally associated with a promoter in an expression vector. Both cDNA and genomic sequences can be cloned and expressed under control of such regulatory sequences. Such vectors can be used to express functional or functionally inactivated TYRO3 subfamily receptor or ligands therefore.

The necessary transcriptional and translational signals can be provided on a recombinant expression vector.

Potential host-vector systems include but are not limited to mammalian or other vertebrate cell systems transfected with expression plasmids or infected with virus, e.g., vaccinia virus, adenovirus, adeno-associated virus, herpes virus, etc.; insect cell systems infected with virus, e.g., baculovirus; microorganisms, such as yeast containing yeast vectors; or bacteria transformed with bacteriophage, DNA, plasmid DNA or cosmid DNA. The expression elements of vectors vary in their strengths and specificities. Depending on the host-vector system utilized, any one of a number of suitable transcription and translation elements may be used.

Expression of a TYRO3 subfamily member polypeptide or ligand thereof may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. Nos. 5,385,839 and 5,168,062), the SV40 early promoter region (see Benoist and Chambon, Nature, Vol. 290, pp. 304-310 (1981), the promoter contained in the 3′ long terminal repeat of Rous sarcoma virus (see Yamamoto, et al., Cell, Vol. 22, pp. 787-797 (1980), the herpes thymidine kinase promoter (see Wagner et al., Proc. Natl. Acad. Sci. USA, Vol. 78, pp. 1441-1445 (1981), the regulatory sequences of the metallothionein gene (see Brinster et al., Nature, Vol. 296, pp. 39-42 (1982); prokaryotic expression vectors, such as the b-lactamase promoter (see Villa-Komaroff et al., Proc. Natl. Acad. Sci. USA, Vol. 75, pp. 3727-3731 (1978) or the tac promoter. See DeBoer et al., Proc. Natl. Acad. Sci. USA, Vol. 80, pp. 21-25 (1983); and also “Useful Proteins from Recombinant Bacteria”, Sci. Am., Vol. 242, pp. 74-94. Still other useful promoter elements which may be used include promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (see Mogram et al., Nature, Vol. 315, pp. 338-340 (1985); and Kollias et al., Cell, Vol. 46, pp. 89-94 (1986)), hematopoletic stem cell differentiation factor promoters, erythropoietin receptor promoter (see Maouche et al., Blood, Vol. 15, p. 2557 (1991)), etc.

In another embodiment, the invention provides methods for expressing TYRO3 subfamily member polypeptides and ligands by using a non-endogenous promoter to control expression of endogenous genes encoding said polypeptides and ligands within a cell. An endogenous gene within a cell is a gene which is ordinarily, i.e., naturally, found in the genome of that cell. A non-endogenous promoter, however, is a promoter or other nucleotide sequence that may be used to control expression of a gene but is not ordinarily or naturally associated with the endogenous gene. As an example, methods of homologous recombination may be employed (preferably using non-protein encoding nucleic acid sequences of the invention) to insert an ampilfiable gene or other regulatory sequence in the proximity of an endogenous gene. The inserted sequence may then be used, e.g., to provide for higher levels of the gene's expression than normally occurs in that cell, or to overcome one or more mutations in the endogenous gene's regulatory sequences which prevent normal levels of gene expression. Such methods of homologous recombination are well known in the art. See, e.g., International Patent Publication No. WO 91/06666, published May 16, 1991 by Skouitchi; International Patent Publication No. WO 91/099555, published Jul. 11, 1991 by Chappel; and International Patent Publication No. WO 90/14092, published Nov. 29, 1990 by Kucherlapati and Campbell.

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE); isoelectric focusing; 2-dimensional gel electrophoresis; chromatography, e.g., ion exchange, affinity, immunoaffinity and sizing column chromatography; centrifugation; differential solubility; immunoprecipitation; or by any other standard technique for the purification of proteins.

Preferred vectors are viral vectors, such as lentiviruses, retroviruses, herpes viruses, adenoviruses, adeno-associated viruses, vaccinia virus, baculovirus and other recombinant viruses with desirable cellular tropism. Thus, a gene encoding a functional or mutant TYRO3 subfamily member polypeptide or ligand, or encoding a domain or fragment thereof, can be introduced in vivo, ex vivo or in vitro using a viral vector or through direct introduction of DNA. Expression in targeted tissues can be effected by targeting the transgenic vector to specific cells, such as with a viral vector or a receptor ligand, or by using a tissue-specific promoter, or both.

Antibodies to TYRO3 Subfamily Member Polypeptides and Ligands Thereto

Antibodies to TYRO3 subfamily member polypeptides and/or ligands are useful, inter alia, for diagnostic and therapeutic methods, as set forth below. According to the invention, TYRO3 subfamily member polypeptides or ligands produced, e.g., recombinantly or by chemical synthesis, and fragments or other derivatives or analogs thereof, including fusion proteins, may be used as an immunogen to generate antibodies that recognize these polypeptides or ligands. Such antibodies include but are not limited to polyclonal, monoclonal, chimeric, single-chain, Fab fragments and an Fab expression library. Such an antibody is preferably specific for a particular TYRO3 subfamily member polypeptide or ligand, i.e., specifically binds to, e.g., a TYRO3 receptor or ligand having the amino acid sequence set forth in SEQ ID NOS:2 and 3, respectively. However, the antibody may, alternatively, be specific for an ortholog from some other species of organism, preferably another species of mammal, such as mouse, rat or hamster, to name a few. The antibody may recognize wild-type, mutant or both forms of a polypeptide or of its ligand.

Various procedures known in the art may be used for the production of polyclonal antibodies. For the production of polyclonal antibodies, various host animals can be immunized by injection with a polypeptide or derivatives, e.g., fragments or fusion proteins, thereof including, but not limited to, rabbits, mice, rats, sheep, goats, etc. In one embodiment, a TYRO3 subfamily polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species including, but not limited to, Freund's (complete and incomplete), mineral gels such as aluminum hydroxide, surface active substances, such as lysolecithin, pluronic polyols, polyanions, peptides, oil emulsions, keyhole limpet hemocyanins, dinitrophenol and potentially useful human adjuvants, such as Bacille Calmette-Guerin (BCG) and Corynebacterium parvum.

For preparation of monoclonal antibodies directed toward a TYRO3 subfamily member polypeptide or ligand, or fragment, analogs, or derivatives thereof, any technique that provides for the production of antibody molecules by continuous cell lines in culture may be used. These include, but are not limited to, the hybridoma technique originally developed by Kohler and Milstein, Nature, Vol. 256, pp. 495-497 (1975), as well as the trioma technique, the human B-cell hybridoma technique (see Kozbor et al., Immunol. Today, Vol. 4, p. 72 (1983); Cote et al., Proc. Natl. Acad. Sci. USA, Vol. 80, pp. 2026-2030 (1983) and the EBV-hybridoma technique to produce human monoclonal antibodies. See Cole et al., Monoclonal Antibodies and Cancer Therapy, Alan R. Liss, Inc., pp. 77-96 (1985). In an additional embodiment of the invention, monoclonal antibodies can be produced in germ-free animals. See International Patent Publication No. WO 89/12690. In fact, according to the invention, techniques developed for the production of “chimeric antibodies” (see Morrison et al., J. Bacterol., Vol. 159, p. 870 (1984); Neuberger et al., Nature, Vol. 312, pp. 604-608 (1984); Takeda et al., Nature, Vol. 314, pp. 452-454 (1985) may also be used. Briefly, such techniques comprise splicing the genes from an antibody molecule from a first species of organism, e.g., a mouse, that is specific for a particular receptor or ligand, together with genes from an antibody molecule of appropriate biological activity derived from a second species of organism, e.g., from a human. Such chimeric antibodies are within the scope of this invention.

Antibody fragments which contain the idiotype of the antibody molecule can be generated by known techniques. For example, such fragments include, but are not limited to, the F(ab′)₂ fragment which can be produced by pepsin digestion of the antibody molecule, the Fab′ fragments which can be generated by reducing the disulfide bridges of the F(ab′)₂ fragment and the Fab fragments which can be generated by treating the antibody molecule with papain and a reducing agent.

According to the invention, techniques described for the production of single-chain antibodies (see U.S. Pat. Nos. 5,476,786, 5,132,405 and 4,946,778) can be adapted to produce specific single-chain antibodies that specifically bind to a particular TYRO3 subfamily member polypeptide or ligand. An additional embodiment of the invention ublizes the techniques described for the construction of Fab expression libraries (see Huse et al., Science, Vol. 246, pp. 1275-1281 (1989)), to allow rapid and easy identification of monoclonal Fab fragments with the desired specificity for a TYRO3 subfamily member polypeptide or ligand, or for its derivatives or analogs.

In the production and use of antibodies, screening for or testing with the desired antibody can be accomplished by techniques known in the art, e.g., radioimmunoassay; enzyme-linked immunosorbant assay (ELISA); “sandwich” immunoassays; immunoradiometric assays; gel diffusion precipitin reactions; immunodiffusion assays; in situ immunoassays, e.g., using colloidal gold, enzyme or radioisotope labels; Western blots; precipitation reactions; agglutination assays, e.g., gel agglutination assays and hemagglutination assays; complement fixation assays; immunofluorescence assays; protein A assays; and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

The foregoing antibodies can be used in methods known in the art relating to the localization and activity of a polypeptide or ligand of interest, e.g., for Western blotting, imaging polypeptides in situ, measuring levels thereof in appropriate physiological samples, etc. using any of the detection techniques mentioned above or known in the art. Such antibodies can also be used in assays for ligand binding to a receptor, e.g., as described in U.S. Pat. No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance for perturbation of optimal conditions, e.g., increasing or decreasing ionic strength, temperature or pH, or adding detergents or chaotropic salts, such perturbations generally decrease binding stability.

In still other embodiments, antibodies may also be used to isolate cells which express a polypeptide or ligand of interest, e.g., OA chondrocyte cells, by panning or related Immunoadsorption techniques.

In a specific embodiment, antibodies that agonize or antagonize the activity of a TYRO3 subfamily member polypeptide can be generated. In particular, intracellular single-chain Fv antibodies can be used to regulate (inhibit) polypeptide activity. See Marasco et al., Proc. Natl. Acad. Sci. USA, Vol. 90, pp. 7884-7893 (1993); Chen, Mol. Med. Today, Vol. 3, pp. 160-167 (1997); Spitz et al., Anticancer Res., Vol. 16, pp. 3415-3422 (1996); Indolfi et al., Nat. Med., Vol. 2, pp. 634-635 (1996); and Kijma et al., Pharmacol. Ther., Vol. 68, pp. 247-267 (1995). Such antibodies can be tested using the assays described infra for Identifying ligands.

Applications and Uses

Described herein are various applications and uses for the TYRO3 subfamily member polypeptides and ligands thereof, including applications and uses for TYRO3, Axl, cMer, GAS6 and PROS1 nucleic acids, polypeptides, and antibodies thereto as described above. In addition, the applications and methods described herein include those that use compounds, e.g., modulators, such as agonists or antagonists that modulate TYRO3 subfamily member polypeptides and ligands therefore, as well as compounds, e.g., modulators, such as antisense and/or inhibitory nucleic adds that modulate the expression of these polypeptides and ligands.

Applicants have determined that the members of the TYRO3 subfamily of receptor tyrosine kinases, as well as ligands thereof, may be used as drug targets in screening assays for the identification of novel therapeutics to treat, prevent or ameliorate OA and other conditions characterized by pathological cartilage degradation and as such methods that utilize TYRO3 subfamily member and related ligand nucleic acids and/or polypeptides in this regard are included herein. For example, methods are described, below, which use compounds that interfere with or modulate binding of a TYRO3 subfamily receptor to a ligand, such as GAS6. In other embodiments, such methods may use compounds that modulate downstream signaling events resulting from the binding of a TYRO3 subfamily member ligand to the appropriate TYRO3 subfamily receptor. Such compounds can be readily identified by persons skilled in the art, e.g., by using screening assays of this invention.

In addition, the TYRO3 subfamily of genes and their gene products, as well as genes and gene product for TYRO3 subfamily ligands, such as GAS6 or PROS1, can also be used as tissue specific markers to detect and/or identify OA cartilage or tissue, such as OA chondrocyte cells. Accordingly, the nucleic acids and polypeptides described, supra, for TYRO3 subfamily receptors and ligand(s) may be used in methods for detecting OA, e.g., in diagnostic and prognostic applications by using TYRO3, Axl and/or cMer genes and gene products (including variants) to detect TYRO3, Axl and/or cMer expression in a sample, such as a tissue sample, e.g., from a biopsy; from an individual. Methods are provided herein that use TYRO3 subfamily member nucleic acids and polypeptides to detect cartilage degradation, such as degradation that is associated with OA and other arthritic conditions.

Drug Screening Assays

Using screening assays, such as those described below, it is possible to identify compounds that bind to or otherwise interact with a TYRO3 subfamily member receptor and/or ligand, including intracellular compounds, e.g., proteins or portions of proteins; compounds that interact with a gene for a TYRO3 subfamily member receptor or its ligand, other natural and synthetic TYRO3 subfamily member ligands or receptors; compounds that interfere with the interaction of a TYRO3 subfamily member gene product, e.g., compounds that interfere with specific binding of a TYRO3 subfamily member gene product to GAS6 or another ligand; and compounds that modulate the activity of a TYRO3 subfamily member gene, e.g., by modulating the level of TYRO3 subfamily member receptor or ligand gene expression; or the activity, e.g., the bioactivity; of a TYRO3 subfamily member receptor or ligand. In this way, such assays may be applied to the identification of compounds which interact with TYRO3, Axl or cMer and their respective ligands.

The screening assays of this invention may therefore be used to identify compounds that specifically bind to a TYRO3 subfamily member gene or gene product and which can thus modulate expression. For example, the screening assays described here may be used to identify compounds that bind to a promoter or other regulatory sequence of a TYRO3 gene, and so may modulate the level of TYRO3 expression. See, e.g., Plaft, J. Biol. Chem., Vol. 269, pp 28558-28562 (1994). The screening assays may also be used to identify compounds that bind to and thereby stabilize a TYRO3 nucleic acid or polypeptide. In addition, these screening assays may be used to identify compounds that inhibit or modulate such binding interactions and which are therefore useful, e.g., as agonists or antagonists for TYRO3 binding to a specific transcription factor or enhancer, or for TYRO3 binding to a stabilizer. Compounds identified in these or similar screening assays may therefore be used to treat diseases and disorders that are associated with abnormal TYRO3 subfamily member gene expression and/or abnormal levels of expressed protein including, but not limited to, OA.

Classes of compounds that may be identified by such screening assays include, but are not limited to, small molecules, e.g., organic or inorganic molecules which are less than about 2 kDa in molecular weight, are more preferably less than about 1 kDa in molecular weight and/or are able to cross the blood-brain barrier or gain entry into an appropriate cell and affect expression of either a TYRO3 subfamily member gene or of some gene involved in the subfamily's regulatory pathway; as well as macromolecules, e.g., molecules greater than about 2 kDa in molecular weight. Compounds identified by these screening assays may also include nucleic acids, peptides and polypeptides. Examples of such compounds (including peptides) include, but are not limited to, soluble peptides; fusion peptide members of combinatorial libraries, such as ones described by Lam et al., Nature, Vol. 354, pp. 82-84 (1991); and by Houghten et al., Nature, Vol. 354, pp. 84-86 (1991); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell, Vol. 72, pp. 767-778 (1993); antibodies including, but not limited to, polyclonal, monoclonal, humanized, anti-idiotypic, chimeric or single-chain antibodies; antibody fragments including, but not limited to, Fab, F(ab′)₂, Fab expression library fragments and epitope-binding fragments thereof. Nucleic acids used in these screening assays may be DNA or RNA, or synthetic nucleic acids. Particular examples include, but are by no means limited to, antisense nucleic acids and ribozymes, as well as double-stranded and triple helix nucleic acid molecules.

Assays for Binding Compounds

In vitro systems can be readily designed to identify compounds capable of binding to the TYRO3 subfamily member gene products of the present invention. Such compounds can be useful, e.g., in modulating the expression, stability or activity of a wild-type TYRO3 gene product or, alternatively, to modulate the expression, stability or activity of a mutant or other variant TYRO3 gene product.

Generally, such screening assays involve preparation of a reactive mixture comprising a TYRO3 subfamily gene product and a test compound under conditions and for a time sufficient to allow the two compounds to interact, e.g., bind, thereby forming a complex that may be detected. The assays may be conducted in any of a variety of different ways. For example, one embodiment comprises anchoring a TYRO3 subfamily polypeptide or a test compound onto a solid phase and detecting complexes of the TYRO3 subfamily polypeptide and the test compound that are on the solid phase at the end of the reaction and after removing, e.g., by washing, unbound compounds. For example, in one preferred embodiment of such a method, a TYRO3 gene product may be anchored onto a solid surface and a labeled compound, e.g., labeled according to any of the methods described supra, is contacted to the surface. After incubating the test compound for a sufficient time and under sufficient conditions that a complex may form between the TYRO3 gene product and the test compound, unbound molecules of the test compound are removed from the surface, e.g., by washing; and labeled molecules which remain are detected.

In another, alternative embodiment, molecules of one or more different test compounds are attached to the solid phase and molecules of a labeled TYRO3 subfamily polypeptide may be contacted thereto. In such embodiments, the molecules of different test compounds are preferably attached to the solid phase at a particular location on the solid phase so that test compounds that bind to the TYRO3 subfamily polypeptide may be identified by determining the location of the bound TYRO3 subfamily polypeptides on the solid phase or surface.

Assays for Compounds that Interact with TYRO3 Subfamily Polypeptides and Ligands

Any of a variety of known methods for detecting protein-protein interactions may also be used to detect and/or identify proteins that interact with a receptor or its ligand. For example, co-immunoprecipitation, cross-linking and co-purification through gradients or chromatographic columns, as well as other techniques known in the art may be employed. Proteins which may be identified using such assays include, but are not limited to, extracellular proteins, such as novel TYRO3 subfamily member ligands, as well as intracellular proteins, such as signal transducing proteins.

Compounds, including other cellular proteins and nucleic acids, that interact with a TYRO3 subfamily member polypeptide and/or ligand may themselves be used in the methods of this invention, e.g., to modulate activity of the TYRO3, Axl or cMer gene or gene product and to treat or prevent cartilage degradation. Alternatively, such interacting compounds may, themselves, be used in the screening assays of this invention to identify other compounds that modulate TYRO3 subfamily member activity, e.g., by binding to TYRO3 ligand and/or downstream signal events resulting therefrom; and could, in turn, be used to treat or prevent cartilage degradation.

As an example, and not by way of limitation, an expression cloning assay may be used to identify novel TYRO3 ligands and other proteins that specifically interact with a TYRO3 receptor. In such assays, a cDNA expression library may be generated from any cell line that expresses a TYRO3-specific ligand, e.g., cells that express GAS6. Clones from such an expression library may then be transfected or infected into cells that do not normally express a TYRO3-specific ligand. Cells that are transfected with a clone that encodes a TYRO3-specific ligand may then express this gene product, and can be Identified and isolated using standard techniques such as FACS or using magnetic beads that have the TYRO3 polypeptide, e.g., an Fc-fusion of the TYRO3 polypeptide, attached thereto.

Alternatively, TYRO3 subfamily member polypeptides and/or ligands may be isolated from a cell line using immunoprecipitation techniques that are well-known in the art.

TYRO3 subfamily member polypeptides and/or ligands may also be isolated using any of the screening assays discussed, supra for identifying binding compounds. For example, a TYRO3-Fc fusion polypeptide may be bound or otherwise attached to a solid surface, and a labeled compound, e.g., a TYRO3 ligand, may be contacted to the surface for a sufficient time and under conditions that permit formation of a complex between the fusion polypeptide and the test compound. Unbound molecules of the test compound can then be removed from the surface, e.g., by washing, and labeled compounds that remain bound can be detected.

Once so isolated, standard techniques may be used to identify any protein detected in such assays. For example, at least a portion of the amino acid sequence of a protein that interacts with a TYRO3, AXL or cMer gene product can be ascertained using techniques well-known in the art, such as the Edman degradation technique. See, e.g., Creighton, Proteins: Structures and Molecular Principles, W.H. Freeman & Co., NY, pp. 34-49 (1983).

Once such proteins have been identified, their amino acid sequence may be used as a guide for the generation of oligonucleotide mixtures to screen for gene sequences encoding such proteins, e.g., using standard hybridization or PCR techniques described supra. See, e.g., Ausubel supra; and PCR Protocols: A Guide to Methods and Applications, Innis et al., Eds., Academic Press, Inc., NY (1990), for descriptions of techniques for the generation of such oligonucleotide mixtures and their use in screening assays.

Other methods are known in the art which may be used in the simultaneous identification of genes that encode a protein that interacts with a TYRO3 subfamily member gene or gene product. For example, expression libraries may be probed with a labeled TYRO3 polypeptide.

As another example and not by way of limitation, a two-hybrid system may be used to detect protein interactions with a TYRO3 subfamily member gene product in vivo. Briefly, utilizing such a system, plasmids may be constructed which encode two hybrid proteins, one of which preferably comprises the DNA-binding domain of a transcription activator protein fused to a TYRO3 (or AXL or cMer as the case may be) gene product. The other hybrid protein preferably comprises an activation domain of the transcription activator protein used in the first hybrid, fused to an unknown protein that is encoded by a cDNA recombined into the plasmid library as part of a cDNA library. Both the DNA-binding domain fusion plasmid and the cDNA library may be co-transformed into a strain of Saccharomyces cerevisiae or other suitable organism which contains a reporter gene, e.g., HBS, lacZ, HIS3 or GFP. Preferably, the regulatory region of this reporter gene comprises a binding site for the transcription activator moiety of the two hybrid proteins. In such a two-hybrid system, the presence of either of the two hybrid proteins alone cannot activate transcription of the reporter gene. Specifically, the DNA-binding domain hybrid protein cannot activate transcription because it cannot localize to the necessary activation function. Likewise, the activation domain hybrid protein cannot activate transcription because it cannot localize to the DNA binding site on the reporter gene. However, interaction between the two hybrid proteins, reconstitutes that functional transcription activator protein and results in expression of the reporter gene. Thus, in a two-hybrid system such as the one described herein, an interaction between a TYRO3 polypeptide, i.e., the TYRO3 polypeptide fused to the transcription activator's DNA binding domain; and a test polypeptide, i.e., a protein fused to the transcription activator's DNA binding domain; may be detected by simply detecting expression of a gene product of the reporter gene.

cDNA libraries for screening in such two-hybrid and other assays may be made according to any suitable technique known in the art. As a particular and non-limiting example, cDNA fragments may be inserted into a vector so that they are translationally fused to the transcriptional activation domain of GAL4, and co-transformed along with a “bait” GAL4 fusion plasmid (encoding a GAL4-fusion of a TYRO3 gene product) into a strain of Saccharomyces cerevisiae or other suitable organism that contains a HIS3 gene driven by a promoter that contains a GAL4 activation sequence. A protein from this cDNA library, fused to the GAL4 transcriptional activation domain, which interacts with the TYRO3 polypeptide moiety of the GAL4-fusion will reconstitute and active GAL4 protein, and can thereby drive expression of the HIS3 gene. Colonies that express the HIS3 gene may be detected by their growth on petri dishes containing semi-solid agar based media lacking histidine. The cDNA may then be purified from these strains, sequenced and used to identify the encoded protein which interacts with the TYRO3 polypeptide.

Once compounds have been identified which bind to a TYRO3 subfamily member gene or gene product of the invention, the screening methods described in these methods may also be used to identify other compounds, e.g., small molecules, peptides and proteins, which bind to these binding compounds. Such compounds may also be useful for modulating bioactivities associated with a TYRO3 subfamily member gene and its gene product, e.g., by binding to a natural ligand and preventing its interaction with the gene product.

Assays for Compounds that Interfere with TYRO3 Subfamily Ligand Interaction

As noted, supra, TYRO3 subfamily members may interact with one or more molecules, e.g., with a specific ligand, in vivo or in vitro. Compounds that disrupt or otherwise interfere with this binding interaction are therefore useful in modulating biological activity or activities that are associated with this subfamily of tyrosine kinases, e.g., tyrosine kinase activity, including, e.g., cartilage degradation. Such compounds may therefore be useful, e.g., to treat, prevent or ameliorate disorders, such as OA that are associated with abnormal levels of TYRO3 expression and/or activity.

Such compounds include, but are not limited to, compounds identified according to the screening assays described, supra, for identifying compounds that bind to TYRO3 subfamily member polypeptides, including any of the numerous exemplary classes of compounds described herein.

In general, assays for identifying compounds that interfere with the interaction between a gene product and a binding partner, e.g., a ligand, involve preparing a test reaction mixture that contains the gene product and its binding partner under conditions and for a time sufficient for the gene product and its binding partner to bind and form a complex. In order to test a compound for inhibitory activity, i.e., for the ability to inhibit formation of the binding complex or to disrupt the binding complex once formed, the test compound preferably is also present in the test reaction mixture. In one exemplary embodiment, the test compound may be initially included in the test reaction mixture with the gene product and its binding partner. Alternatively, however, the test compound may be added to the test reaction mixture at a later time, subsequent to the addition of the gene product and its binding partner. In preferred embodiments, one or more control reaction mixtures, which do not contain the test compound, may also be prepared. Typically, a control reaction mixture will contain the same gene product and binding partner that are in the test reaction mixture, but will not contain a test compound. A control reaction mixture may also contain a placebo, not present in the test reaction mixture, in place of the test compound. The formation of a complex between the gene product and the binding partner may then be detected in the reaction mixture. The formation of such a complex in the absence of the test compound, e.g., in a control reaction mixture, but not in the presence of the test compound, indicates that the test compound is one which interferes with or modulates the interaction of the gene product and its binding partner.

Such assays for compounds that modulate the interaction of a gene product and a binding partner may be conducted in a heterogenous format or, alternatively, in a homogeneous format. Heterogeneous assays typically involve anchoring either a gene product or a binding partner onto a solid phase and detecting compounds anchored to the solid phase at the end of the reaction. Thus, such assays are similar to the solid phase assays described, supra, for detecting and/or identifying nucleic acids and gene products and for detecting or identifying ligands. Indeed, those skilled in the art will recognize that many of the principles and techniques described above for those assays may be modified and applied without undue experimentation in the solid phase assays described here, for identifying compounds that modulate interaction(s) between a gene product and a binding partner.

Regardless of the particular assay used, the order to which reactants are added to a reaction mixture may be varied, e.g., to identify compounds that interfere with the interaction of a TYRO3 subfamily member gene product with a binding partner by competition, or to identify compounds that disrupt a preformed binding complex. Compounds that interfere with the interaction of a gene product with a binding partner by competition may be identified by conducting the reaction in the presence of a test compound. Specifically, in such assays a test compound may be added to the reaction mixture prior to or simultaneously with the gene product and the binding partner. Test compounds that disrupt preformed complexes of a gene product and a binding partner may be tested by adding the test compound to a reaction mixture after complexes have been formed.

The screening assays described herein may also be practiced using peptides or polypeptides that correspond to portions of a full-length TYRO3, Axl or cMer polypeptide or protein, or with fusion proteins comprising such peptide or polypeptide sequences. For example, screening assays for identifying compounds the modulate interactions of a TYRO3 polypeptide with a binding partner may be practiced using peptides or polypeptides corresponding to particular regions or domains of a full-length TYRO3 polypeptide that bind to a binding partner, e.g., receptor “binding sites”.

A variety of methods are known in the art that may be used to identify specific binding sites of a TYRO3 subfamily member polypeptide. For example, binding sites may be identified by mutating a TYRO3 gene and screening for disruptions of binding as described above. A gene encoding the binding partner may also be mutated in such assays to identify mutations that compensate for disruptions from the mutation to the TYRO3 gene. Sequence analysis of these mutations can then reveal mutations that correspond to the binding region of the two proteins.

In an alternative embodiment, a protein, e.g., a TYRO3 protein or a protein binding partner to a TYRO3 protein, may be anchored to a solid surface or support using the methods described hereinabove. Another labeled protein which binds to the protein anchored to the solid surface may be treated with a proteolytic enzyme, and its fragments may be allowed to interact with the protein attached to the solid surface, according to the methods of the binding assays described, supra. After washing, short, labeled peptide fragments of the treated protein may remain associated with the anchored protein. These peptides can be isolated and the region of the full-length protein from which they are derived may be identified by the amino acid sequence.

In still other embodiments, compounds that interfere with interactions between a TYRO3 subfamily member polypeptide and ligand thereto may also be identified by screening for compounds that modulate binding of the polypeptide, e.g., an Fc-fusion construct of the TYRO3 polypeptide, to cells that express a specific ligand thereto.

Diagnostic and Prognostic Applications

A variety of methods can be employed for diagnostic and prognostic applications using reagents, such as the TYRO3 subfamily member nucleic acids and polypeptides described, supra, as well as antibodies directed against such nucleic acids and polypeptides. For example, using the methods described herein it is possible to detect expression of a TYRO3 subfamily nucleic acid or protein in a biological sample from an individual, such as in cells or tissues in a sample, e.g., from a biopsy, obtained or derived from an individual subject or patient. Preferred cells or tissues used for such applications are those involved in OA, e.g., chondrocyte cells or articular joint tissue(s), such as cartilage, synovial fluid and/or serum. As explained above, both TYRO3 receptor and its ligand GAS6 are expressed at elevated levels in such OA cells and tissue.

Thus, using the methods described herein, as well as other methods known in the art, a skilled artisan may detect elevated levels of a TYRO3 or GAS6 nucleic acid or polypeptide in a sample of cells or tissue from an individual, and may thereby detect and/or identify cells or tissue in that sample as being symptomatic of OA. In certain preferred embodiments the particular type of tissue identified in such methods is cartilage tissue. By using such methods to detect such cells or tissue in an individual, a skilled user may thereby diagnose the presence of OA in that individual. In preferred embodiments the methods described herein are performed using prepackaged diagnostic kits. Such kits may comprise at least one specific TYRO3 nucleic acid or a TYRO3 gene product specific antibody reagent. For example, said diagnostic kit may be used for detecting mRNA levels or protein levels of a TYRO3 subfamily member gene or gene product, said kit comprising: (a) a polynucleotide of a TYRO3 subfamily member or a fragment thereof; (b) a nucleotide sequence complementary to that of (a); (c) an expression product of said TYRO3 subfamily member gene, or a fragment thereof; or (d) an antibody to said expression product and wherein components (a), (b), (c) or (d) may comprise a substantial component.

In preferred embodiments, a kit will also contain instructions for its use, e.g., to detect diseased cells or tissues, or to diagnose a disorder, such as OA, associated with abnormal expression of a TYRO3 gene or gene product. In preferred embodiments, such instructions may be packaged directly with the kit. In other embodiments, however, instructions may be provided separately. For example, the invention provides embodiments of kits where instructions for using the kit may be downloaded, e.g., from the internet A kit of the invention may also comprise, preferably in separate containers, suitable buffers and other solutions to use the reagents, e.g., nucleic acid or antibody specific for a TYRO3 gene or gene product, to detect the TYRO3 gene or gene product. The kit and any reagent(s) contained therein may be used, e.g., in a clinical setting, to diagnose patients exhibiting or suspected of having OA.

A sample comprising a cell of any cell type or tissue of any tissue type in which a TYRO3 gene is expressed may also be used in such diagnostic methods, e.g., for detection of TYRO3 gene expression or of TYRO3 gene products, such as TYRO3 polypeptides, as well as for identifying cells, e.g., chondrocytes, that express a TYRO3 gene or a TYRO3 gene product. Thus, in one embodiment, the methods described herein may be performed in situ, e.g., using cells or tissues obtained from an individual, such as in a biopsy.

The methods described herein are not limited to diagnostic applications, but may also be used in prognostic applications, e.g., to monitor the progression of a disease, such as OA, that is associated with abnormal expression of a TYRO3 subfamily gene or gene product, or to monitor a therapy thereto. Accordingly, prognostic methods of the invention may comprise, in one exemplary embodiment, monitoring TYRO3 nucleic acid or polypeptide levels in an individual during the course of a treatment or therapy, e.g., a drug treatment or exercise regimen, for OA. Similarly, the methods of the invention may also be used to detect and identify diseased cells and tissue, e.g., cells over-expressing TYRO3 compared to non-OA cells or tissue, during the course of a therapy. In such embodiments, decreasing numbers of diseased cells is generally indicative of an effective treatment. The methods of the invention may further be used, e.g., to screen candidate drugs or compounds and identify ones that may be effective, e.g., as anti-OA drugs. Such methods may be performed in vivo, e.g., using an animal model, or in vitro, e.g., in a cell culture assay. In one embodiment such methods may comprise contacting a test compound to a cell and identifying whether expression of a TYRO3 gene or gene product by the cell has been inhibited. In another embodiment, a test compound may be contacted to a cell or administered to an organism, and extracellular levels of TYRO3 nucleic acid or polypeptide may be measured, e.g., in cell culture media for cell culture assays, or in tissue, blood or other body fluid in an animal model assay.

Detection of TYRO3 Subfamily Nucleic Acids

The diagnostic and prognostic methods of the invention include methods for assaying the level of TYRO3 subfamily, preferably TYRO3, gene expression. A variety of methods known in the art may be used to detect assay levels of nucleic acid sequences in a sample. For example, RNA from a cell type or tissue that is known or suspected to express a particular gene may be isolated and tested utilizing hybridization or PCR techniques known in the art. The isolated cells may be, e.g., cells derived from a cell culture or from an individual. The analysis of cells taken from a cell culture may be useful, e.g., to test the effect of compounds on the expression of a gene, or alternatively, to verify that the cells are ones of a particular cell type that express a gene of interest.

As an example, and not by way of limitation, diagnostic methods for the detection of, e.g., TYRO3 nucleic acids can involve contacting and incubating nucleic acids (including recombinant DNA molecules, cloned genes or degenerate variants thereof) obtained from a sample with one or more labeled nucleic acid reagents, such as recombinant TYRO3 DNA molecules, cloned genes or degenerate variants thereof, under conditions favorable for specifically annealing or hybridizing these reagents to their complementary sequences in the sample nucleic acids. After incubation, all non-annealed or non-hybridized nucleic acids are removed. The presence of nucleic acids that have hybridized, if any such molecules exist, is then detected and the level of TYRO3 nucleic acid sequences to which the nucleic acid reagents have annealed may be compared to the annealing pattern or level expected from a control sample, e.g., from a sample of normal, non-OA cells or tissues, to determine whether TYRO3 nucleic acid is expressed at an elevated level.

In a preferred embodiment of such a detection scheme, the nucleic acid from the cell type or tissue of interest may be immobilized, e.g., to a solid support such as a membrane or a plastic surface, e.g., on a nylon membrane, a microtiter plate or on polystyrene beads. After incubation, non-annealed, labeled TYRO3 subfamily nucleic acid reagents may be easily removed and detection of the remaining, annealed, labeled TYRO3 subfamily nucleic acid reagents may be accomplished using standard techniques that are well-known in the art.

Alternative diagnostic methods for the detection of TYRO3 subfamily nucleic acids in patient samples or in other cell or tissue sources may involve their amplification, e.g., by PCR, see, e.g., the experimental embodiment taught in U.S. Pat. No. 4,683,202, followed by detection of the amplified molecules using techniques that are well-known to those skilled in the art. The resulting level of amplified TYRO3 subfamily nucleic acids may be compared to those levels that would be expected if the sample being amplified contained only normal levels of the TYRO3 subfamily nucleic acid(s), as normal cells or tissues, to determine whether elevated levels of any TYRO3 subfamily nucleic acid(s) are expressed, e.g., levels in a healthy chondrocyte.

In one preferred embodiment of such a detection scheme, a cDNA molecule is synthesized from an RNA molecule of interest, e.g., by reverse transcription. A sequence within the cDNA may then be used as a template for a nucleic acid amplification reaction, such as PCR. Nucleic acid reagents used as synthesis initiation reagents, e.g., primers, in the reverse transcription and amplification steps of such an assay are preferably chosen from the TYRO3 subfamily nucleic acid sequences described herein or are fragments thereof. Preferably, the nucleic acid reagents are at least about 9-30 nucleotides in length. The amplification may be performed using, e.g., radioactively labeled or fluorescently labeled nucleotides, for detection. Alternatively, enough amplified product may be made such that the product can be visualized by standard ethidium bromide or other staining methods.

TYRO3 subfamily gene expression assays of the invention may also be performed in situ, i.e., directly upon tissue sections of patient tissue, which may be fixed and/or frozen, thereby eliminating the need for nucleic acid purification. TYRO3 subfamily nucleic acid reagents may be used as probes or as primers for such in situ procedures. See, e.g., Nuovo, PCR In Situ Hybridization: Protocols And Application, Raven Press, NY (1992). Alternatively, if a sufficient quantity of the appropriate cells can be obtained, standard Northern analysis can be performed to determine the level of TYRO3 subfamily gene expression by detecting levels of TYRO3 subfamily mRNAs.

Detection of TYRO3 Subfamily Member Gene Products

The diagnostic and prognostic methods of the invention also include ones that comprise detecting levels of a TYRO3 subfamily member protein or other polypeptide, and including functionally conserved variants and fragments thereof. For example, antibodies directed against unimpaired, wild-type or mutant TYRO3 (or Axl or cMer) gene products or against functionally conserved variants or peptide fragments of a TYRO3 gene product may be used as diagnostic and prognostic reagents. Such reagents may be used, e.g., to detect abnormalities in the level of TYRO3 gene product synthesis or expression, or to detect abnormalities in the structure, temporal expression or physical location of a TYRO3 subfamily gene product. Antibodies and immunoassay methods such as those described herein also have important in vitro applications for assessing the efficacy of treatments, e.g., for OA. For example, antibodies or fragments of antibodies, can be used in screens of potentially therapeutic compounds in vitro to ascertain a compound's effects on TYRO3 expression and TYRO3 polypeptide production. Compounds that may have beneficial effects on a disorder associated with abnormal TYRO3 subfamily expression can be identified and a therapeutically effective dose for such compounds may be determined using such assays.

As one example, antibodies or fragments of antibodies may be used to detect the presence of a TYRO3 subfamily gene product, a variant of said gene product or fragments thereof, e.g., by immunofluorescence techniques employing a fluorescently labeled antibody coupled with light microscopic, flow cytometric or fluorimetric detection methods.

In particularly preferred embodiments, antibodies or fragments thereof may also be employed histologically, e.g., in immunofluorescence or immunoelectron microscopy techniques, for in situ detection of a TYRO3 subfamily member gene product. In situ detection may be accomplished by removing a histological specimen, e.g., a tissue sample, from a patient and applying thereto a labeled antibody of the present invention or a fragment of such an antibody. The antibody or antibody fragment is preferably applied by overlaying the labeled antibody or antibody fragment onto a biological sample. Through the use of such a procedure, it is possible to detect, not only the presence of, e.g., a TYRO3 subfamily gene product, but also the gene product's distribution in the examined tissue. A wide variety of histological methods that are well-known in the art, e.g., staining procedures, can be readily modified by those skilled in the art without undue experimentation to achieve such in situ detection.

Immunoassays useful for identifying gene products will typically comprise incubating a biological sample, e.g., a tissue extract, in the presence of a detectably labeled antibody that is capable of specifically binding a gene product of interest including, e.g., a functionally conserved variant or a peptide fragment thereof. The bound antibody may then be detected by any of a number of techniques well-known in the art.

Therapeutic Methods and Pharmaceutical Compositions

TYRO3 subfamily member nucleic acids and polypeptides, modulators (e.g. agonists, antagonists, inhibitors) thereof and specific antibodies thereto may also be used in therapeutic methods and compositions, e.g., to treat, prevent or ameliorate diseases and disorders associated with abnormal (preferably elevated) levels of the TYRO3 expression e.g., OA, used as a pharmaceutical or in the manufacture of a medicament.

Accordingly, in certain preferred embodiments the therapeutic methods of the present invention comprise administering a pharmaceutical composition comprising one or more compounds or modulators that modulate, e.g., inhibit, TYRO3 subfamily member expression or activity, e.g., compounds that bind to a TYRO3 subfamily member nucleic acid or polypeptide of the invention, compounds that modulate expression of a TYRO3 subfamily member gene, and/or compounds that interfere with or modulate binding of a TYRO3 subfamily member nucleic acid or polypeptide with a binding partner, such as a TYRO3 subfamily member-specific ligand, e.g., PROS1 or GAS6.

In another preferred embodiment, the therapeutic methods of the invention may comprise one or more cell-targeted therapies which target compounds, e.g., drugs, pro-drugs, toxins or cytotoxins, to cells expressing a TYRO3 subfamily member nucleic acid or polypeptide.

Inhibitory Approaches

In alternative embodiments, the present invention provides methods and compositions for treating a disease or disorder, e.g., OA, associated with the abnormal expression or activity of a TYRO3 subfamily member gene or gene product by modulating, e.g., increasing or decreasing, the expression or activity of the TYRO3 subfamily member gene or its gene product. Such methods may simply comprise administering one or more compounds that modulate expression, synthesis, or activity of, e.g., a TYRO3, AXL or cMer gene. Preferably, these one or more compounds are administered until one or more symptoms of the disorder are eliminated or at least ameliorated.

Among the compounds that may exhibit an ability to modulate the activity, expression or synthesis of a TYRO3 subfamily member nucleic acid of interest are antisense molecules. Such molecules may be designed to reduce or inhibit wild-type nucleic acids and polypeptides or, alternatively, may target mutant nucleic acids or polypeptides. Antisense molecules may also be used to inhibit the expression of nucleic acids for a ligand.

Antisense RNA and DNA molecules act to directly block the translation of mRNA by hybridizing to target mRNA molecules and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required. As used in this description, “antisense” broadly includes RNA-RNA interactions, triple helix interactions, ribozymes and RNase-H mediated arrest. Antisense nucleic acid molecules can be encoded by a recombinant gene for expression in a cell (see, e.g., U.S. Pat. Nos. 5,814,500 and 5,811,234) or, alternatively, they can be prepared synthetically (see U.S. Pat. No. 5,780,607).

A sequence that is “complementary” to a portion of a nucleic acid refers to a sequence having sufficient complementary to be able to hybridize with the nucleic acid and form a stable duplex. The ability of nucleic acids to hybridize will depend both on the degree of sequence complementary and the length of the antisense nucleic acid. Generally, however, the longer the hybridizing nucleic acid, the more base mismatches it may contain and still form a stable duplex (or triplex in triple helix methods). A tolerable degree of mismatch can be readily ascertained, e.g., by using standard procedures to determine the melting temperature of a hybridized complex.

In one preferred embodiment, oligonucleotides complementary to non-coding regions of a TYRO3 subfamily member gene may be used in an antisense approach to inhibit translation of the endogenous TYRO3 subfamily member mRNA molecules. Antisense nucleic acids are preferably at least 6 nucleotides in length, and more preferably range from between about 6 to about 50 nucleotides in length. In specific embodiments, the oligonucleotides may be at least 10, at least 15, at least 20, at least 25 or at least 50 nucleotides in length.

It is generally preferred that in vitro studies are first performed to quantitate the ability of an antisense oligonucleotide to inhibit gene expression. It is preferred that these studies utilize controls that distinguish between antisense gene inhibition and non-specific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target mRNA sequence.

While antisense nucleotides complementary to any portion of the target gene coding region sequence could be used, those complementary to the transcribed, untranslated region are most preferred.

Antisense molecules are preferably delivered to cells, such as chondrocytes, that express the target gene in vivo. A number of methods have been developed for delivering antisense DNA or RNA to cells. For example, antisense molecules can be injected directly into the tissue site, e.g., directly into a tumor, or modified antisense molecules can be designed to target the desired cells, e.g., antisense linked to peptides or antibodies that specifically bind receptors or antigens expressed on the target cell surface, can be administered systemically.

Preferred embodiments achieve intracellular concentrations of antisense nucleic acid molecules which are sufficient to suppress translation of endogenous mRNAs. For example, one preferred approach uses a recombinant DNA construct in which the antisense oligonucleotide is placed under the control of a strong pol III or pol II promoter. The use of such a construct to transfect target cells will result in the transcription of sufficient amounts of single-stranded RNAs that will form complementary base pairs with the endogenous target gene transcripts and thereby prevent translation of the target gene mRNA. For example, a vector, as set forth above, can be introduced, e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral or others known in the art, used for replication and expression in mammalian cells. Expression of the sequence encoding the antisense RNA can be by any promoter known in the art to act in the particular cell type, e.g., in a hemopoietic cell. For example, any of the promoters discussed, supra, in connection with the expression of recombinant TYRO3 subfamily member nucleic adds can also be used to express a TYRO3 subfamily member antisense nucleic acid.

In addition to antisense technology, RNA aptamers (see Good et al., Gene Ther., Vol. 4, pp. 45-54 (1997), double-stranded RNA (WO 99/32619), ribozymes (see Cech, Amer. Med Assn., Vol. 260, p. 3030 (1988); Cotton et al., EMBO J., Vol. 8, pp. 3861-3866 (1989); Grassi and Marini, Ann. Med., Vol. 28, pp. 499-510 (1996); and Gibson, Cancer Metast Rev., Vol. 15, pp. 287-299 (1996); and/or triple helix DNA (see Gee et al., Molecular and Immunologic Approaches, Huber and Carr, Eds., Futura Publishing Co., Mt. Kisco, N.Y. (1994), may be used to modulate the activity, expression or synthesis of a target nucleic acid according to methods familiar to one of skill in the art.

Alternatively, small interfering RNA (siRNA) molecules can also be used to inhibit the expression of nucleic acids for a polypeptide or ligand of interest RNA interference is a method in which exogenous, short RNA duplexes are administered where one strand corresponds to the coding region of the target mRNA. See Elbashir et al., Nature, Vol. 411, pp. 494-498 (2001). Upon entry into cells, siRNA molecules cause not only degradation of the exogenous RNA duplexes, but also of single-stranded RNAs having identical sequences, including endogenous messenger RNAs. Accordingly, siRNA may be more potent and effective than traditional antisense RNA methodologies since the technique is believed to act through a catalytic mechanism.

Preferred siRNA molecules are typically greater than about 19 nucleotides in length and comprise the sequence of a nucleic acid for a TYRO3 subfamily member or its ligand. Effective strategies for delivering siRNA to target cells include any of the methods described, supra, for delivering antisense nucleic acids. For example, siRNA can be introduced to cells by transduction using physical or chemical transfection. Alternatively siRNAs may be expressed in cells using, e.g., various PolIII promoter expression cassettes that allow transcription of functional siRNA or precursors thereof. See, e.g., Scherr et al., Curr. Med. Chem., Vol. 10, No. 3, pp. 245-256 (2003); Turki et al., Hum. Gene Ther., Vol. 13, No. 18, pp. 2197-2201 (2002); and Cornell et al., Nat. Struct. Biol., Vol. 10, No. 2, pp. 91-92 (2003).

Pharmaceutical Preparations

Compositions used in the therapeutic methods of this invention may be administered, e.g., in vitro or ex vivo to cell cultures, or, more preferably, in vivo to an individual, at therapeutically effective doses to treat a disease or disorder such as OA that is associated with abnormal TYRO3 subfamily gene expression and/or activity. For example, compounds, including compounds identified in such screening methods as described above, that bind to a TYRO3 subfamily gene or gene product of the invention may be administered to the cells or individual so that expression and/or activity of the gene or gene product is inhibited. The invention therefore also provides pharmaceutical preparations for use, e.g., as therapeutic compounds to treat disorders, including OA, that are associated with abnormal TYRO3 subfamily gene expression or activity.

The terms “therapeutically effective dose” and “effective amount” refer to the amount of the compound that is sufficient to result in a therapeutic response. In embodiments where a compound, e.g., a drug or toxin, is administered in a complex, e.g., with a specific antibody, the terms “therapeutically effective dose” and “effective amount” may refer to the amount of the complex that is sufficient to result in a therapeutic response. A therapeutic response may be any response that a user, e.g., a clinician, will recognize as an effective response to the therapy. Thus, a therapeutic response will generally be an amelioration of one or more symptoms of a disease or disorder. In preferred embodiments, where the pharmaceutical preparations are used to treat OA, a therapeutic response may be a reduction in the amount of cartilage degradation observed, e.g., in biopsies from a patient during treatment.

Toxicity and therapeutic efficacy of compounds can be determined by standard pharmaceutical procedures, e.g., in cell culture assays or using experimental animals to determine the LD₅₀ and the ED₅₀. The parameters LD₅₀ and ED₅₀ are well-known in the art, and refer to the doses of a compound that are lethal to 50% of a population and therapeutically effective in 50% of a population, respectively. The dose ratio between toxic and therapeutic effects is referred to as the therapeutic index and may be expressed as the ratio: LD₅₀/ED₅₀. Compounds that exhibit large therapeutic indices are preferred.

While compounds that exhibit toxic side effects may be used, however, in such instances it is particularly preferable to use delivery systems that specifically target such compounds to the site of affected tissue so as to minimize potential damage to other cells, tissues or organs and to reduce side effects.

Data obtained from cell culture assay or animal studies may be used to formulate a range of dosages for use in humans. The dosage of compounds used in therapeutic methods of the present invention preferably lie within a range of circulating concentrations that includes the ED₅₀ concentration but with little or no toxicity, e.g., below the LD₅₀ concentration. The particular dosage used In any application may vary within this range, depending upon factors, such as the particular dosage form employed, the route of administration utilized, the conditions of the individual, e.g., patient, and so forth.

A therapeutically-effective dose may be initially estimated from cell culture assays and formulated in animal models to achieve a circulating concentration range that includes the IC₅₀. The IC₅₀ concentration of a compound is the concentration that achieves a half-maximal inhibition of symptoms, e.g., as determined from the cell culture assays. Appropriate dosages for use in a particular individual, e.g., in human patients, may then be more accurately determined using such information.

Measures of compounds in plasma may be routinely measured in an individual, such as a patient by techniques, such as high performance liquid chromatography (HPLC) or gas chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, e.g., tablets or capsules prepared by conventional means with pharmaceutically acceptable excipients, such as binding agents, e.g., pregelatinised maize starch, polyvinylpyrrolidone or hydroxypropyl methylcellulose; fillers, e.g., lactose, microcrystalline cellulose or calcium hydrogen phosphate; lubricants, e.g., magnesium stearate, talc or silica; disintegrants, e.g., potato starch or sodium starch glycolate; or wetting agents, e.g., sodium lauryl sulphate. The tablets may be coated by methods well-known in the art. Liquid preparations for oral administration may take the form of, e.g., solutions, syrups or suspensions, or they may be presented as a dry product for constitution with water or other suitable vehicle before use. Such liquid preparations may be prepared by conventional means with pharmaceutically acceptable additives such as suspending agents, e.g., sorbitol syrup, cellulose derivatives or hydrogenated edible fats; emulsifying agents, e.g., lecithin or acacia; non-aqueous vehicles, e.g., almond oil, oily esters, ethyl alcohol or fractionated vegetable oils; and preservatives, e.g., methyl or propyl-p-hydroxybenzoates or sorbic acid. The preparations may also contain buffer salts, flavoring, coloring and sweetening agents, as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base, such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents, such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions, such as suppositories or retention enemas, e.g., containing conventional suppository bases, such as cocoa butter or other glycerides.

In addition to the formulations described previously, the compounds may also be formulated as a depot preparation. Such long acting formulations may be administered by implantation, e.g., subcutaneously or intramuscularly; or by intramuscular injection. Thus, e.g., the compounds may be formulated with suitable polymeric or hydrophobic materials, e.g., as an emulsion in an acceptable oil; or ion exchange resins, or as sparingly soluble derivatives, e.g., as a sparingly soluble salt.

The compositions may, if desired, be presented in a pack or dispenser device that may contain one or more unit dosage forms containing the active ingredient. The pack may, e.g., comprise metal or plastic foil, such as a blister pack. The pack or dispenser device may be accompanied by instructions for administration.

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited and discussed In this specification (including references to biological sequences deposited in GenBank or other public databases) are incorporated herein by reference in their entirety and to the same extent as if each reference was individually incorporated by reference.

### EXAMPLES

The present invention is also described by means of the following examples. However, the use of these or other examples anywhere in the specification is illustrative only and in no way limits the scope and meaning of the invention or of any exemplified term. Likewise, the invention is not limited to any particular preferred embodiments described herein. Indeed, many modifications and variations of the invention may be apparent to those skilled in the art upon reading this specification and can be made without departing from its spirit and scope.

### Example 1

### OA Marker Genes

The following materials and methods are used to perform the examples described below:

Preparation of Plasmid DNA from Full Length cDNA Clones

Bacterial stocks containing full-length TYRO3 OA cDNA in pCMVSport6 vector are grown in 96 deep-well blocks (Qiagen, Valencia, Calif.), each well containing 1.0 mL of Terrific broth (Sigma, St. Louis, Mo.) and ampicillin (40 μg/mL). The cultures are initially grown for 24 hours at 37° C. with shaking at 300 RPM, re-innoculated into a fresh block and further grown overnight to ensure uniform growth of bacteria in all wells. Plasmid DNA is isolated from the bacteria with a Biorobot 8000 (Qiagen, Valencia, Calif.) following standard protocols described by the manufacturer.

GATEWAY™ Transfer of Full-Length cDNA Clones.

In order to test TYRO3 in an RT-PCR assay, TYRO3 cDNA is transferred from the pCMVSport6 vector to a retroviral vector using the GATEWAY™ platform (Invitrogen, Carlsbad, Calif.).

GATEWAY BP reactions are carried out as follows. Briefly, 1.0 μL (100-120 ng) plasmid DNA is added to a microtitre well containing 1 μL (100-120 ng) pDONR 201 entry vector (Invitrogen, Carlsbad, Calif.), 1 μL BP reaction buffer (Invitrogen, Carlsbad Calif.), 1 μL tris-EDTA and 1 μL BP Clonase enzyme mix (Invitrogen, Carlsbad, Calif.) on ice. The microtitre plates is incubated at 25° C. for 3 hours.

The GATEWAY LR reaction mix, consisting of 0.25 μL of 0.75 M NaCl, 1.0 μL (100-120 ng) linearized retroviral vector and 1.5 μL LR Clonase enzyme mix (Invitrogen, Carlsbad, Calif.) is added to the BP reaction. The retroviral vector used in this study contains a hybrid CMV/Moloney murine leukemia virus 5′LTR, a Moloney murine leukemia virus 3′LTR and a retroviral packaging ψ site and was constructed according to conventional methods. The same vector is also commercially available (Clontech). The sample is mixed thoroughly and incubated for 2 additional hours at 25° C. One-tenth volume (0.8 μL; 2 mg/mL) of Proteinase K solution (Invitrogen, Carlsbad, Calif.) is added and incubated at 37° C. for 10 minutes.

Forty μL of max efficiency DH5α cells (Invitrogen, Carlsbad, Calif.) are aliquoted into wells of a flat bottom 96-well block (Qiagen, Valencia, Calif.) on ice. One μL of the LR reaction mixture is then added to the cells and incubated on ice for 30 minutes. Cells are heat shocked for 30 seconds at 42° C., placed on ice for 1-2 minutes, and 65 μL of S.O.C. medium (Invitrogen, Carlsbad, Calif.) is added to each well. The 96-well block is incubated at 37° C. for 1 hour with shaking. Thirty-five μL of the final transformation mixture is added to a microtitre well containing LB agar with 40 μg/mL zeocin (Invitrogen, Carlsbad, Calif.), and grown overnight at 37° C.

Single colonies are inoculated to 1 mL Terrific broth/zeocin (40 μg/mL) in 96-well format and grown overnight at 37° C. in a rotary shaker at 300 RPM. Plasmid DNA is isolated using a Biorobot 8000 (Qiagen, Valencia, Calif.) following standard protocols described by the manufacturer.

Production of Supernatants

GP2-293 packaging cells (BD Biosciences Clontech, Palo Alto, Calif.) are seeded (5×10⁴ cells/well) in 96-well PDL plates (BD Biosciences Clontech, Palo Alto. CA) 16-24 hours prior to transfection in antibiotic-free DMEM containing 10% FBS (Invitrogen, Carlsbad, Calif.). GATEWAY™ constructs along with envelope vector pVPack-VSV-G (Stratagene, La Jolla, Calif.) are co-transfected into the packaging cells by combining 150 ng GATEWAY™ construct with 150 ng envelope plasmid in a total volume of 25 μL OPTIMEM (Invitrogen, Calsbad, Calif.) in a 96-well format In a separate plate, 25 μL of OPTIMEM™ is combined with 1 μL of Lipofectamine 2000 reagent (Invitrogen, Carlsbad, Calif.). This second solution is incubated for 5 minutes at room temperature, and the two solutions are then combined. The DNA-lipofectamine complex is allowed to form for 20 minutes before being added to the cells. The media is replaced with complete media containing antibiotics 16-24 hours after the transfection procedure. The media, containing viral supernatants, is collected at 24 and 48 hours post-transfection.

Transduction into Primary Chondrocytes

Primary chondrocytes (isolated from cartilage tissue obtained from joint replacement surgery, Mullenberg Hospital, Plainfield, N.J.) are seeded at 1.1×10⁴ cells/well in duplicate 96-well plates, 24 hours prior to transduction. At time of transduction, media are replaced with 100 μL viral supernatant and 100 μL complete media supplemented with 20 mM HEPES and 16 μg/mL polybrene. Cells are centrifuged in a swinging bucket rotor at 32° C., 1000×g, for 1.5 hours. The media are replaced after 16-24 hours with fresh media, and cells are incubated for an additional 48 hours.

RNA Isolation and RT-PCR

Total cellular RNA is isolated from pooled duplicate 96-well plates using a BioRobot 8000 (Qiagen, Valencia, Calif.) and Qiagen RNeasy 96 Biorobot reagents according to the manufacturer's instructions. On-column DNase I digestion is employed, pursuant to standard protocols published by Qiagen (Valencia, Calif.) to eliminate contaminating genomic DNA. First strand cDNA is synthesized using random primers with a High-Capacity cDNA Archive kit (PE Applied Biosystems, Foster City Calif.) in a 100 μL reaction volume. Real time PCR (RT-PCR) was performed in a 384-well format on the ABI Prism 7900HT Sequence Detection System (Applied Biosystems, Foster City, Calif.). The cDNA template and PCR mix are distributed using a Biomek FX liquid handling robot. The 20 μL reaction contains 5 μL cDNA, 200 nM forward and reverse primers, and SYBR Green PCR Master Mix (Applied Biosystems, Foster City, Calif.). The default cycling program (95° C.-10 minutes and 40 cycles of 95° C.-15 seconds, 60° C.-1 minute) is followed by a dissociation stage whereby a melting curve is generated to confirm the specificity of the PCR product and the absence of primer dimers.

Table I below specifies exemplary OA “marker genes” familiar to one of skill in the art. In particular, each of the “marker genes” used in this assay is a gene associated with OA in chondrocyte cells. The GenBank Accession Number for an exemplary nucleotide sequence is also provided for each marker gene.

RT-PCR experiments use the primers specified in Table I (designed with Primer Express software (Applied Biosystems, Foster City, Calif.) under default parameters and reaction conditions) to amplify each of the genes listed. In addition, the gene GAPDH (GenBank Accession No. AJ 005371) is selected as a ubiquitously-expressed “housekeeping” gene to which all samples are normalized.

TABLE I
RT-PCR Primers to Detect OA Marker Genes

| | | |
|---|---|---|
| Marker Gene | Primer | Sequence |

| | | | |
|---|---|---|---|
| Aggrecanase-1 | Forward | 5′-TTTCCCTGGCAAGGACTATGA-3′ | (SEQ ID NO:5) |
| | Reverse | 5′-AATGGCGTGAGTCGGGC-3′ | (SEQ ID NO:6) |
| MMP-13 | Forward | 5-TGATCTCTTTTGGAATTAAGGAGCAT-3′ | (SEQ ID NO:7) |
| | Reverse | 5′-ATGGGCATCTCCTCCATAATTTG-3′ | (SEQ ID NO:8) |
| COX-2 | Forward | 5-AAATTGCTGGCAGGGTTGC-3′ | (SEQ ID NO:9) |
| | Reverse | 5′-TTTCTGTACTGCGGGTGGAAC-3′ | (SEQ ID NO:10) |
| INOS | Forward | 5-GCAAACCTTCAAGGCAGCC-3′ | (SEQ ID NO:11) |
| | Reverse | 5′-TGCTGTTTGCCTCGGACAT-3′ | (SEQ ID NO:12) |
| Collagen IIa | Forward | 5′-ACGCTGCTCGTCGCCG-3′ | (SEQ ID NO:13) |
| | Reverse | 5′-GCCAGCCTCCTGGACATCCT-3′ | (SEQ ID NO:14) |
| Collage X | Forward | 5-ACCCAACACCAAGACACAGTTCT-3′ | (SEQ ID NO:15) |
| | Reverse | 5′-TCTTACTGCTATACCTTTACTCTTTATGGTGTA-3′ | (SEQ ID NO:16) |
| Collagen I | Forward | 5′-CAGCCGCTTCACCTACAGC-3′ | (SEQ ID NO:17) |
| | Reverse | 5′-TTTTGTATTCAATCACTGTCTTGCC-3′ | (SEQ ID NO:18) |
| Decorin | Forward | 5′-GCCAGCCTCCTGGACATCCT-3′ | (SEQ ID NO:19) |
| | Reverse | 5′-AGTCCTTTCAGGCTAGCTGCATC-3′ | (SEQ ID NO:20) |
| Aggrecan | Forward | 5′-TCGAGGACAGCGAGGCC-3′ | (SEQ ID NO:21) |
| | Reverse | 5′-TCGAGGGTGTAGCGTGTAGAGA-3′ | (SEQ ID NO:22) |
| Gapdh | Forward | 5′-ATGGGGAAGGTGAAGGTCG-3′ | (SEQ ID NO:23) |
| | Reverse | 5′-TAAAAGCAGCCCTGGTGACC-3′ | (SEQ ID NO:24) |

ROX dye (Applied Biosystems, Foster City, Calif.) is used as a passive reference to normalize non-PCR related fluctuations in the fluorescence signal. Changes in gene expression are calculated according to the manufacturers instructions (Applied Biosystems, Foster City, Calif.) using the comparative Cₜ method which makes use of a calibrator sample, i.e., a sample to which all others are compared. The value of the calibrator sample is normalized as 1.0 so that expression levels for all other samples are defined as multiples of the expression level measured for the calibrator sample. For RT-PCR experiments described in this example, a retroviral vector containing no cDNA insert is used as the calibrator sample. Briefly, the amount of target mRNA relative to the calibrator mRNA is calculated according to the formula: 2^{-ΔΔC}^{_{T}} where Ct=thresh hold cycle (cycle#at which the amount of amplified target reaches a fixed threshold).

Cell Treatment

To optimize RT-PCR conditions and validate the markers chosen in this test, human articular chondrocytes from knee joint cartilage obtained in joint replacement surgeries were plated in 96-well plates (11,000 cells/well) using DMEM medium containing 10% FBS (Invitrogen, Carlsbad, Calif.). Two days later, the cells are treated with IL-1 (5 ng/mL; Peprotech, UK, London) and OSM (50 ng/mL) or PDGF (50 ng/mL) or TGF-β (50 ng/mL) overnight in serum free medium. OSM, PDGF and TGF-β are purchased from R&D systems, (Minneapolis, Minn.). RNA is isolated from these cells and evaluated by RT-PCR using the methods described above.

ELISA

Levels of MMP13 and IL-6 protein in cell culture supernatants are determined using a human MMP13 ELISA system from Amersham (Piscataway, N.J.) and a human IL-6 ELISA system from R&D Systems (Minneapolis, Minn.) following the manufacturers' standard protocols.

Results

In order to identify drug targets useful for the treatment of OA, the effects in chondrocytes of candidate gene overexpression on several know genetic markers of OA was observed. Table II below summarizes exemplary OA marker genes and related OA characteristic.

TABLE II
OA Marker Genes

| OA Characteristic | Marker Gene | Accession No. |
|---|---|---|
| Cartilage degradation | AGGRECANASE-1 | [AF148213] |
| | MMP-13 | [XM_006274] |
| Aberrant chondrocyte cell | COLLAGEN TYPE I | [AF017178] |
| differentiation (hypertrophy | COLLAGEN TYPE IIA | [XM_012271] |
| and proliferation) | COLLAGEN TYPE X | [NM_000493] |
| Inflammation | INOS | [AB022318] |
| | COX-2 | [M90100] |
| Matrix synthesis | AGGRECAN | [X80278] |
| | DECORIN | [AF491944] |

Before candidate genes (in this case TYRO3) are transfected into chondrocytes and effect of expression on OA marker genes assessed, the primers and RT-PCR conditions to be used with the specific OA marker genes are validated. This is done by assaying changes in expression of OA marker genes in chondrocytes treated with various compounds known to induce OA characteristics.

Specifically, human articular chondrocyte cells are treated with various cytokines and growth factors described in the materials and methods, above, known to induce an OA characteristics in chondrocyte cells. See Tardif et al., Arthritis Rheum., Vol. 42, No. 6, pp. 1147-1158 (1999); and Smith et al., Arthritis Rheum., Vol. 34, No. 6, pp. 697-706 (1991). Using the primer described herein, RT-PCR is then performed to determine whether there is any detectable change in expression of one or more marker genes. Table III, below, summarizes exemplary changes in mRNA levels of each marker mediated by treatment of the chondrocyte cells with: (i) IL-1 and OSM; (ii) TGF-β; and (iii) PDGF. Expression levels are indicated as the multiples of normalized expression levels, i.e., as the “fold changes” in mRNA levels, measured in untreated chondrocyte cells.

The data in Table III indicates that the various OA marker genes undergo changes in their expression levels that would be expected in response to known treatments that induce OA characteristics in chondrocytes. Thus, these data validate the usefulness of the primers and methodologies employed.

TABLE III
Change of Marker Gene Expression in Treated Chondrocyte Cells

Treatment

| Marker Gene | IL-1/OSM | TGF-B | PDGF | Untreated |
|---|---|---|---|---|
| Aggrecanase-1 | 50.21 | 3.81 | 2.46 | 1.00 |
| MMP-13 | 125.37 | 6.92 | 4.20 | 1.00 |
| Collagen IIa | −227.54 | 1.45 | −2.04 | 1.00 |
| Collagen X | −3.71 | 19.97 | −1.79 | 1.00 |
| Collagen I | −3.58 | 3.84 | −1.89 | 1.00 |

To further validate the RT-PCR assay, the constitutively active gene product AKT/PKB (GenBank Accession No. NPL-001907) is overexpressed in chondrocyte cells by retroviral-mediated gene transfer (method as described in Example 1). Activation of this gene's biochemical pathway is known to induce Aggrecanse-1 and MMP-13 in chondrocyte cells. Using conventional methods, cellular RNA is harvested 48 hours and 72 hours post-transduction, and changes in the expression of MMP-13 and Aggrecanse-1 mRNA are detected by RT-PCR. AKT/PKB over-expression results in a 12-fold induction of Aggrecanase-1 and a 9-fold induction of MMP-13 (data not shown).

### Example 2

### TYRO3

In order to determine what role, if any, TYRO 3 has in OA, a full-length TYRO3 cDNA is transfected into primary human articular chondrocyte (HAC) cells and the resulting effect on the expression of genetic markers of OA in these cells is analyzed by RT-PCR, and compared to the expression levels of those marker genes measured in untransformed cells.

Results indicate that the transfection of the TYRO3 gene potently induces the expression of at least four OA marker genes, suggesting that TYRO3 is involved in the OA disease process (see Table IV, below).

TABLE IV
TYRO3 Induces a Plurality of OA Genetic Markers

| | |
|---|---|
| Gene | Fold Induction of MRNA |

| Description | Aggrecanase-1 | MMP-13 | INOS | COX-2 |
|---|---|---|---|---|
| TYRO3 | 51 | 29 | 400 | 15 |

To verify that TYRO3 induced an elevated expression of polypeptides encoded by these “marker” genes, MMP-13 protein levels were measured by ELISA in chondrocyte cells transformed with the TYRO3 cDNA. Results at indicate that TYRO3 over-expression does lead to the induction of MMP-13 protein secretion, as measured by ELISA in the cell supernatant (data not shown).

### Example 3

### GAS6

This example describes experiments that investigate the ability of GAS6, a ligand that binds to and activates TYRO3 subfamily members, to induce OA marker genes in chondrocyte cells.

The following materials and methods were used to perform the examples described below:

Immunohistochemical Analysis

Full thickness explants of normal or OA human knee cartilage are cultured in DMEM containing 5% FBS. Cartilage is fixed with 4% paraformaldehyde and embedded with paraffin to cut 5 micron sections. The tissue sections are placed on slides, deparaffinized in toluene, hydrated in graded series of ethanol, then washed in PBS and 0.2% peroxidase. After blocking the tissue sections with normal serum or BSA for 30 minutes, the sections are incubated with primary antibody against GAS6 (goat anti-human recombinant antibody from R&D Systems, Minneapolis, Minn.) or antibody to human TYRO3 (goat antibody to human TYRO3 extracellular domain, from R&D Systems, Minneapolis, Minn.) at 2 μg/mL for 1-2 hours at room temperature or overnight at 4° C. This TYRO3 antibody does not cross-react with Axl or cMer.

After washing sections 3 times for 5 minutes in PBS, a second blocking reaction is performed for 10 minutes. The sections are incubated for 30 minutes with diluted biotinylated secondary antibody. The slides are washed 3 times in PBS and incubated for 30 minutes with Vectastain ABC-AP (Vector Labs, Burlingame, Calif.) or the peroxidase-based Elite ABC system (Vector Labs, Burlingame, Calif.). The slides are washed and the section incubated for 4-20 minutes with alkaline phosphatase substrate solution (Vector Labs, Burlingame, Calif.) or with 3,3-diaminobenzidine (DAB) substrate. Slides are rinsed with water, counterstained with diluted Hematoxylin or Methyl Green, rehydrated in graded ethanol or in three changes of 1-butanol, hemo-de and mounted with Refrax mounting medium (Anatech Ltd., Battle Creek, Mich.). Negative controls were performed by replacing the primary antibody with pre-immune serum or immunoglobulin.

Production of Recombinant GAS6

To generate GAS6 protein, a full-length GAS6 DNA clone from an in-house clone collection is transfected into 293H cells (Invitrogen, Carlsbad, Calif.) using Lipofectamine 2000 (Invitrogen, Carlsbad, Calif.) following the manufacturer's recommended protocol. A preferred cDNA sequence for GAS6 and a protein sequence that encodes the GAS6 polypeptide are available from GenBank under the Accession numbers NM_{—}00820 (SEQ ID NO:28) and NP_{—}000811 (SEQ ID NO:29), respectively. GAS6 protein is secreted by transformed cells into the supernatant and collected 72 hours post-transfection.

Chondrocyte Treatment

Chondrocytes are plated in 24-well plates and treated overnight with either straight or serially diluted 293H cell supernatant containing recombinant GAS6 protein. Total RNA is isolated and RT-PCR is performed to detect expression of Aggrecanase-1, MMP-13 and INOS following the RT-PCR methods described in Example 1, above. Alternatively, ELISA is performed for the proteins MMP-13 or IL-6, following the materials and methods for ELISA described above.

Results

TYRO3 and GAS6 are Overexpressed in Human Joint Disease

Immunohistochemistry is used to examine TYRO3 and GAS6 levels directly in normal and OA human cartilage tissue in order to determine whether TYRO3 and its ligand, GAS6 are actually over-expressed in human joint disease. Results indicate the presence of both TYRO3 and GAS6 in OA cartilage but not in normal human cartilage tissue. Moreover, cultured mid and deep zone chondrocyte cells from OA patients exhibit increased immue staning for TYRO3 and GAS6 (data not shown). These data support the conclusion that OA chondrocytes are activated in vivo in an autocrine fashion because of increased expression of (at least) TYRO3 and GAS6. Thus, both TYRO3 and GAS6 are important mediators of OA in vivo and can be used, e.g., as drug targets to treat such disorders.

GAS6 Induces OA Marker Genes and Gene Products

In order to determine whether endogenous TYRO3 may be activated using recombinant GAS6 and whether such activation leads to induction of marker genes (proteinases and inflammatory mediators) indicative of OA chondrocyte cells are treated with 293H cell supernatants expressing GAS6 as described above. Results indicate that such treatment leads to the induction of Aggrecanase-1, MMP-13 and iNOS, as determined by RT-PCR. By contrast, supernatants from 293H cells transfected with an empty vector do not induce these genes. ELISA experiments also demonstrate that treatment of chondrocytes with GAS6-containing supernatant also induces the expression of IL-6 protein (data not shown).

The results from these experiments indicate that not only does treatment of chondrocyte cells with GAS6 induce the expression of OA marker genes but also, like TYRO3, GAS6 is expressed at elevated levels in OA chondrocytes.

### Example 4

### Inhibition of TYRO3 Reduces the Expression of OA Marker Genes

This example describes experiments that use RNA interference to specifically inhibit TYRO3 gene expression in chondrocyte cells.

The following materials and methods are used for the example below:

Primary human articular chondrocyte cells (isolated from cartilage tissue obtained from knee joint replacement surgery, Mullenberg hospital, Plainsfield, N.J.) are seeded 24 hours prior to transfection at 30-50% confluence (approximately 1.5×1 cells/well). Cells are washed once to remove traces of antibiotics and 350 μL of OPTIMEM (Invitrogen, Carlsbad, Calif.) is added to each well. Three μL of 20 μM siRNA duplex (Dharmacon, Lafayette, Colo.) is added to 50 μL of Opti-MEM (Invitrogen, Carlsbad, Calif.). In a separate tube, 3 μL of Oligofectamine (Invitrogen, Carlsbad Calif.) is diluted Into 12 μL Opti-MEM and incubated for 9 minutes. The siRNA duplex solution and the oligofectamine solutions are combined and incubated for another 20 minutes. The volume of resting solution is adjusted to 100 μL and added to the chondrocyte cells such that the final concentration of oligonucleotide duplex in each well is 133 nM and the Oligofectamine concentration is 0.67%.

Cells are incubated for 16 hours with the transfection media at which time the media is switched to 10% heat inactivated serum (Invitrogen, Carlsbad, Calif.) in DMEM (Invitrogen, Carsbad, Calif.) containing antibiotics (Invitrogen, Carlsbad, Calif.). The cells are incubated an additional 48 hours to silence the target genes. Cells are then challenged with IL-1 or TNF (5 ng/mL and 50 ng/mL, respectively) for 16 hours in DMEM containing 10% iron supplemented calf serum (Omega Scientific, Tarzana, Calif.). The media is collected and assayed for MMP-13 production (MMP-13 ELISA system, Amersham, Piscataway, N.J.). RNA is isolated from the cell pellets (Qiagen RNeasy, Valencia, Calif.) and message levels of selected genes are measured by RT-PCR as described, supra.

The following siRNA oligonucleotide
sequences are used:

| | |
|---|---|
| Target | |
| Gene | Sirna Sequence |

| | | |
|---|---|---|
| Human TNF | 5-AACCAAGUGCCACAAAGGAAC-3′ | (SEQ ID NO:25) |
| Receptor | | |
| TYRO3 | 5′-AAACAUCGAGAGAGCUGAGGA-3′ | (SEQ ID NO:26) |
| Luci- | 5′-AAGUGCGCUGCUGGUGCCAAC-3′ | (SEQ ID NO:27) |
| ferase | | |

*From pGL3 vector (Promega, Madison, WI).

Results

RNA interference is a method by which post transcription expression of particular genes may be selectively inhibited, so that the function of one or more specific genes may be evaluated in the context of the cell. In these experiments, chemically synthesized double-stranded 21 mers are delivered into cells. These oligomers activate normal cellular processes leading to highly specific degradation of particular mRNA(s).

In this example, siRNA is administered to OA chondrocyte cells, as described above, to inhibit TYRO3 expression. mRNA levels for IL-6 and MMP-13, two genes the expression of which is known to be upregulated in OA chondrocytes (see Vincenti et al., Arthritis Res., Vol. 4, No. 3, pp. 157-164 (2002); Bau et al., Arthritis Rheum., Vol. 46, No. 10, pp. 2648-2657; Flannery et al., Matrix Biol., Vol. 18, No. 3, pp. 225-237 (1999); and Mengshol et al., Arthritis Rheum., Vol. 43, No. 4, pp. 801-811 (2000), are measured by RT-PCR, as are TYRO3 mRNA levels, 48 hours after administering the TYRO3-specific siRNA. Results indicate that the basal expression levels of both IL-6 and MMP-13 are effectively-reduced along with TYRO3 expression. As a negative control, OA chondrocyte cells are also treated with siRNA that specifically inhibits the luciferase gene, a gene not normally expressed in the chondrocyte cells. As expected, administration of this luciferase-specific siRNA does not inhibit the expression of either TYRO3,IL-6 or MMP-13, as determined by RT-PCR.

Still other experiments demonstrate the effect that silencing TYRO3 has on IL-1 mediated effects in OA chondrocytes. Specifically, chondrocyte cells are treated with TYRO3-specific siRNA as described above, followed by treatment with either IL-1 or TNF. mRNA levels for MMP-13 and IL-6 are measured by RT-PCR. Data indicate that siRNA inhibition of TYRO3 effectively inhibits the expression of both MMP-13 and IL-6 mRNA that is normally induced by TNF and/or IL-1. These findings are confirmed by ELISA measurements of MMP-13 protein in the chondrocyte cells. Again, inhibition of TYRO3 effectively inhibits the secretion of MMP-13 protein that is normally induced by IL-1 and/or TNF suggesting that TYRO3 signalling may have an important role in modulating IL-1 and/or TNF induction of catabolic enzymes in chondrocytes. This implicates an important role for TYRO 3 signalling in the pathogenesis of OA.

### Example 5

### AXL, cMer and OA

As explained above, TYRO3 has considerable sequence and structural homology to at least two other receptor tyrosine kinases, Axl and cMer, and these three genes comprise a subfamily of receptor tyrosine kinases. Thus, in addition to TYRO3, cMer and Axl are also expected to be useful in the compositions and methods of the present invention, e.g., to diagnose and/or treat, prevent or ameliorate OA or as drug-targets for compounds that are themselves useful for diagnosing and/or treating OA.

At least two splice variants of the Axl gene are known, one of which (referred to here as “Axl variant 2” or “Axlv2”) lacks an exon (exon 10) that is present in one or more other Axl splice variants. An exemplary Axlv2 nucleic acid sequence is available from GenBank Accession No. NM_{—}001699 and is also provided here at SEQ ID NO:30. An exemplary Axlv2 polypeptide, which is encoded by the Axlv2 nucleic acid at SEQ ID NO:30, comprises an amino acid sequence that is also available from GenBank Accession No. NP_{—}001690 and is set forth here in SEQ ID NO:31. Another Axl splice variant is known, which is referred to here as “Axl variant 1” or “Axlv1”. An exemplary Axlv1 nucleic acid comprises the nucleotide sequence set forth here in SEQ ID NO:32 and in GenBank Accession No. NM_{—}021913. This nucleic acid encodes an Axlv1 polypeptide comprising the amino acid sequence set forth in GenBank Accession No. NP_{—}068713 (SEQ ID NO:33).

In experiments that employ identical methodologies as those described, supra, for TYRO3 (see Examples above) increased expression of an Axlv1 nucleic acid (in house cDNA library) in chondrocyte cells increases the expression of Agg-1 and MMP-13 mRNA by factors of approximately 12 and 15, respectively. Similarly, increased expression of an Axlv2 nucleic acid (in house cDNA library) in chondrocyte cells increases the expression of these same OA “marker” genes, i.e., of Agg-1 and MMP-13, by approximately 3-fold and 8-fold, respectively.

In addition, Axl and cMer si RNA (Dharmacon, Lafayette, Colo.) (delivered into chondrocytes according to the methods used in Example 4) blocked IL-1 mediated induction of Aggrecanase-1 and MMP-13 mRNA in chondrocytes suggesting that all 3 members of this subfamily of receptor tyrosine kinases may have an important role in the pathogenesis of OA.

As such, cMer and AXL (including variants) can be used in the methods and compositions of this invention in the same manner as TYRO3 (and variants thereof) according to the methods and compositions of this invention and, accordingly, such uses are considered a part of the present invention.

What is claimed is:

1. A method for identifying a compound for treating OA, which method comprises:
a) contacting a test compound to a reaction mixture that comprises
(i) a polypeptide member of the TYRO3 subfamily of receptor tyrosine kinases, and
(ii) a ligand to said polypeptide, wherein the reaction mixture conditions permit binding of the polypeptide to the ligand to form a binding complex;
b) detecting levels of formation of the binding complex in the reaction mixture in the presence of the test compound; and
c) comparing the level of the binding complex formed in the presence of the test compound to the level of binding complex formed in the absence of said test compound, wherein a decrease in the level of the binding complex formed in the presence of the test compound indicates that the test compound may be used to treat OA.

2. A method according to claim 1, wherein said polypeptide member of the TYRO3 subfamily of receptor tyrosine kinases is selected from the group consisting of TYRO3, Axl and cMer.

3. A method for identifying a compound for treating osteoarthritis, which method comprises:
a) contacting a test compound to a reaction mixture that comprises
(i) a TYRO3 polypeptide; and
(ii) a TYRO3 ligand, wherein the reaction mixture conditions permit binding of the TYRO3 polypeptide to the TYRO3 ligand to form a binding complex;
b) detecting levels of formation of the binding complex in the reaction mixture in the presence of the test compound; and
c) comparing the level of the binding complex formed in the presence of the test compound to the level of binding complex formed in the absence of said test compound, wherein a decrease in the level of the binding complex formed in the presence of the test compound indicates that the test compound may be used to treat OA.

4. A method according to claim 3, wherein the TYRO3 polypeptide comprises the amino acid sequence set forth in SEQ ID NO:2.

5. A method according to claim 3, in which the TYRO3 ligand is selected from the group consisting of PROS1 and GAS6 polypeptide.

6. A method according to claim 5, wherein the GAS6 polypeptide comprises the amino acid sequence set forth in SEQ ID NO:3.

7. A method for identifying an individual having osteoarthritis, which method comprises:
a) detecting a nucleic acid encoding a polypeptide member of the TYRO3 subfamily of receptor tyrosine kinases in a biological sample derived from the individual; and
b) comparing the level of said nucleic acid in the individual to levels of said nucleic acid in individuals not having osteoarthritis, wherein elevated levels of said nucleic acid in said biological sample derived from the individual indicates that the individual has OA.

8. A method according to claim 7, wherein said polypeptide member of the TYRO3 subfamily of receptor tyrosine kinases is selected from the group consisting of TYRO3, Axl and cMer.

9. A method according to claim 7, wherein said biological sample comprises biological material selected from the group consisting of chondrocytes, cartilage, synovial fluid and serum.

10. A method for identifying an individual having osteoarthritis, which method comprises:
a) detecting a TYRO3 nucleic acid in a biological sample derived from the individual; and
b) comparing the level of the TYRO3 nucleic acid in the individual to levels of the TYRO3 nucleic acid in individuals not having OA, wherein elevated levels of the TYRO3 nucleic acid in said biological sample derived from the individual indicates that the individual has OA.

11. A method according to claim 10, wherein the TYRO3 nucleic acid encodes a polypeptide having the amino acid sequence set forth in SEQ ID NO:2.

12. A method according to claim 10, wherein the TYRO3 nucleic acid comprises the nucleotide sequence set forth in SEQ ID NO:1.

13. A method according to claim 10, wherein said biological sample comprises biological material selected from the group consisting of chondrocytes, cartilage, synovial fluid and serum.

14. A method for identifying an individual having osteoarthritis, which method comprises:
a) detecting levels of a polypeptide of the TYRO3 subfamily of receptor tyrosine kinases in a biological sample derived from the individual; and
b) comparing the level of said polypeptide in the individual to levels of said polypeptide in individuals not having OA, wherein elevated levels of said polypeptide in said biological sample derived from the individual indicates that the individual has OA.

15. A method according to claim 14, wherein said polypeptide of the TYRO3 subfamily of receptor tyrosine kinases is selected from the group consisting of TYRO3, Axl and cMer.

16. A method according to claim 14, wherein said biological sample comprises biological material selected from the group consisting of chondrocytes, cartilage, synovival fluid and serum.

17. A method for identifying an individual having OA, which method comprises:
a) detecting a TYRO3 polypeptide in a biological sample derived from the individual; and
b) comparing the level of the TYRO3 polypeptide in the individual to levels of the TYRO3 polypeptide in individuals not having OA, wherein elevated levels of the TYRO3 polypeptide in said biological sample derived from the individual indicates that the individual has OA.

18. A method according to claim 17, wherein the TYRO3 polypeptide comprises the amino acid sequence set forth in SEQ ID NO:2.

19. A method according to claim 15, wherein said biological sample comprises biological material selected from the group consisting of chondrocytes, cartilage, synovival fluid and serum.

20. A method for identifying an individual having osteoarthritis, which method comprises:
a) detecting, in a biological sample derived from the individual, a nucleic acid that encodes a ligand of a polypeptide member of the TYRO3 subfamily of receptor tyrosine kinases; and
b) comparing the level of the nucleic acid in the individual to levels of the nucleic acid in individuals not having OA, wherein elevated levels of the nucleic acid in said biological sample derived from the individual indicates that the individual has OA.

21. A method according to claim 20, wherein said polypeptide member of the TYRO3 subfamily of receptor tyrosine kinases is selected from the group consisting of TYRO3, Axl and cMer.

22. A method according to claim 20, wherein said biological sample comprises biological material selected from the group consisting of chondrocytes, cartilage, synovival fluid and serum.

23. A method for identifying an individual having osteoarthritis, which method comprises:
a) detecting, in a biological sample derived from the individual, a nucleic acid that encodes a TYRO3 ligand; and
b) comparing the level of the nucleic acid in the individual to levels of the nucleic acid in individuals not having OA, wherein elevated levels of the nucleic acid in said biological sample derived from the individual indicates that the individual has OA.

24. A method according to claim 23, wherein the TYRO3 ligand is selected from the group consisting of PROS1 and GAS6.

25. A method according to claim 23, wherein the TYRO3 ligand comprises the amino acid sequence set forth in SEQ ID NO:3.

26. A method according to claim 23, wherein the nucleic acid encoding said TYRO3 ligand comprises the nucleotide sequence set forth in SEQ ID NO:4.

27. A method according to claim 23, wherein said biological sample comprises biological material selected from the group consisting of chondrocytes, cartilage, synovival fluid and serum.

28. A method for identifying an individual having OA, which method comprises:
a) detecting levels of a ligand of a member of the TYRO3 subfamily of receptor tyrosine kinases in a biological sample derived from the individual; and
b) comparing the level of said ligand in the individual to levels of said ligand in individuals not having OA, wherein elevated levels of said ligand in said biological sample derived from the individual indicates that the individual has OA.

29. A method according to claim 25, wherein said member of the TYRO3 subfamily of receptor tyrosine kinases is selected from the group consisting of TYRO3, Axl and cMer.

30. A method according to claim 25, wherein said biological sample comprises biological material selected from the group consisting of chondrocytes, cartilage, synovival fluid and serum.

31. A method according to claim 25, wherein said ligand is selected from the group consisting of PROS1 and GAS6.

32. A method according to claim 25, wherein said ligand comprises the amino acid sequence set forth in SEQ ID NO:3.

33. A method according to claim 25, wherein a nucleic acid encoding said ligand comprises the nucleotide sequence set forth in SEQ ID NO:4.

34. A method for identifying an individual having osteoarthritis, which method comprises:
a) detecting the level of a TYRO3 ligand in a biological sample derived from the individual; and
b) comparing said level of the TYRO3 ligand in the individual to levels of the TYRO3 ligand in individuals not having OA, wherein elevated levels of the TYRO3 ligand in said biological sample derived from the individual indicates that the individual has OA.

35. A method according to claim 34, wherein the TYRO3 ligand is selected from the group consisting of PROS1 and GAS6.

36. A method according to claim 34, wherein the TYRO3 ligand comprises the amino acid sequence set forth in SEQ ID NO:3.

37. A method according to claim 34, wherein a nucleic acid encoding said TYRO3 ligand comprises the nucleotide sequence set forth In SEQ ID NO:4.

38. A method according to claim 34, wherein said biological sample comprises biological material selected from the group consisting of chondrocytes, cartilage, synovial fluid and serum.
